(19) United States
(12) Patent Application Publication
(10) Pub. No.: US 2020/0224200 A1
(43) Pub. Date: Jul. 16, 2020

(54) MODULATION OF IMMUNE RESPONSE THROUGH STIMULATOR OF INTERFERON GENES

(71) Applicant: La Jolla Institute for Allergy and Immunology, La Jolla, CA (US)

(72) Inventors: Sonia SHARMA, La Jolla, CA (US); Mariko TAKAHASHI, La Jolla, CA (US)

(21) Appl. No.: 16/650,614

(22) PCT Filed: Sep. 25, 2018
(86) PCT No.: PCT/US2018/052713

§ 371 Date: Mar. 25, 2020

Related U.S. Application Data
(60) Provisional application No. 62/563,607, filed on Sep. 26, 2017.

(51) Int. Cl. C12N 15/113; A61K 31/4439; A61K 31/519; A61K 31/7084; A61K 31/713

(52) U.S. Cl. CPC C12N 15/1135; A61K 31/4439; A61K 31/519; A61K 31/7084; A61K 45/06; C12N 15/1137; C12N 2310/14; C12N 2320/12; C12N 2310/531; A61K 31/713

(57) ABSTRACT

Embodiments of the disclosure relate to modulation of an immune response comprising modulating the interaction between a death associated protein kinase (DAPK) and stimulator of interferon genes protein (STING) pathway. In certain embodiments, a method is provided for treating a subject for an inflammatory or autoimmune disease, or cancer, or a side effect or symptom thereof by administering an agent that modulates the interaction between a DAPK and the STING pathway.

### CROSS-REFERENCE TO RELATED PATENT APPLICATION

This application claims priority under 35 U.S.C. § 119(e) to U.S. Provisional Patent Application No. 62/563,607, filed Sep. 26, 2017, the contents of which is hereby incorporated by reference in its entirety.

### STATEMENT OF GOVERNMENT SUPPORT

This invention was made with government support under Contract No. R01CA199376 awarded by the National Institutes of Health. The government has certain rights in the invention.

### BACKGROUND

The innate immune system utilizes pattern recognition receptors (PRRs) to induce a rabid cell-autonomous immune response against invasion of microbial pathogens and cellular and tissue damage. As the first line of self-defense, PRRs including Toll-like receptors (TLRs), Nod-like receptors (NLRs), RIG-I-like receptors (RLRs) recognizes pathogen-associated molecular patterns (PAMPs) or damage-associated molecular patterns (DAMPs), followed by the activation of multiple signaling cascades including nuclear factor-κB (NFκB), mitogen-activated protein kinase (MAPKs), and interferon regulatory factors (IRFs), ultimately lead to the production of pro-inflammatory cytokines, chemokines and type I interferons (IFNs) (Kanneganti (2010); Kawai (2010); Bowie (2008)).

Methods of modulating the innate immune system address a variety of diseases. For example, enhancement of innate immunity can help remediate viral infections and cancers, whereas downregulation of innate immunity can help mitigate the effects of inflammatory and/or autoimmune diseases. Thus, there is a need in the art to identify targets and agents that modulate the innate immune system.

### SUMMARY

Disclosed herein is a method of modulating an immune response, the method comprising, or alternatively consisting essentially of, or yet further consisting of, modulating the interaction between a death associated protein kinase (DAPK) and stimulator of interferon genes protein (STING) pathway. In some embodiments, the method comprises modulating the immune response in a subject, that is in need of the method. In certain embodiments, the immune response is an innate immune response.

Aspects of the disclosure relate to a method of enhancing an innate immune response in a subject comprising, or consisting of, or alternatively consisting essentially of, administering an effective amount of an agonist of the interaction between DAPK and stimulator of interferon genes protein (STING) pathway.

Further aspects relate to a method of increasing type I interferon (IFN-I) expression in a population of cells or tissue comprising, or consisting of, or alternatively consisting essentially of, administering an effective amount of an agonist of the interaction between DAPK and STING pathway to a population of cells or tissue.

Still further aspects relate to a method of downregulating an innate immune response in a subject comprising, or consisting of, or alternatively consisting essentially of, administering an effective amount of an antagonist of the interaction between DAPK and STING pathway.

Additional aspects of this disclosure relate to a method of decreasing type I interferon (IFN-I) expression in a population of cells, the method comprising, or consisting of, or alternatively consisting essentially of, administering an effective amount of an antagonist of the interaction between DAPK and STING pathway to a population of cells.

Other aspects disclosed herein relate to kits comprising an agonist or antagonist disclosed herein and instructions for use according to the corresponding method.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIGS. 1A-1I show the results of a tumor suppressor gene (TSG) siRNA screen to identify DAPK3 as a regulator of DNA sensing pathway. FIG. 1A shows the results of an experiment in which primary HUVECs were stimulated with poly (dA:dT) (1 ug/ml) (right panel) for 4 h, and IRF3 translocation was quantified by immunofluorescence microscopy. Cells were counterstained with DAPI and Phalloidin (AF488). FIG. 1B provides a schematic representation of the siRNA screen targetd tumor suppressor genes (TSGs). FIG. 1C shows the TSG screen (for 1002 genes) results in mean z-score rank order, from decreased IRF3 translocation to increased IRF3 translocation. Primary HUVECs were transfected with siDAPK3 pool, siSTING pool or siControl. 72 h later, cells were stimulated with poly (dA:dT) (0.5 ug/ml), VACV70 (2 ug/ml), poly I:C (1 ug/ml), or infected with hCMV, vesicular stroma virus(VSV), and Sendai Virus (SeV) at an MOI of 5; the data is provided in FIGS. 1D and 1E. To generate FIG. 1D, IRF3 nuclear translocation was quantified after 3 h or 6 h for virus infection, and, to generate FIG. 1E, level of IFNb mRNA was quantified by RT-qPCR. FIG. 1F shows relative expression levels of DAPK family in human cell lines (left panel) and mouse cell lines (right panel) were estimated by real time (RT)-qPCR. To generate FIG. 1G, THP1-ISG cells transduced with lentiviral shDAPK3 (#1 and #2), lentiviral shSTING (#1 and #2), or shControl were stimulated with poly (dA:dT) (0.5 ug/ml), VACV70 (2 ug/ml), poly I:C (1 ug/ml) for 4 h. mRNA levels of IFNb, CXCL10, and IL6 were quantified after 4 h stimulation. L929-mRuby-hIRF3 transduced with lentiviral shDAPK3 (#1 and #2), lentiviral shSTING (#1 and #2), or shControl were stimulated with poly (dA:dT) (0.5 ug/ml), VACV70 (2 ug/ml), poly I:C (1 ug/ml) for 4 h; the data is provided in FIGS. 1H and 1I. To generate FIG. 1H, IRF3 translocation were examined at 3 h after stimulation and, to generate FIG. 1I, mRNA levels of IFNb, IFIT1, CXCL10, and MX2 were quantified after 4 h stimulation.

FIGS. 2A-2F show that DAPK3 is required for DNA-stimulated transcription factor activation and ISG induction. L929-mRuby-hIRF3 cells were transfected with DAPK3-targeting siRNAs, cGAS-targeting siRNA, STING-targeting siRNA, or non-targeting control siRNA (siCtrl). 72 h later, cells were stimulated with 2′3-cGAMP (10 μM) and the percent IRF3 nuclear translocation was calculated from values for cells transfected with control siRNA. The results are shown in FIGS. 2A and 2B. To generate FIG. 2A, Cells were stimulated with poly dA:dT (0.5 ug/ml) for 12 h and intracellular cGAMP level was quantified by mass spectrometry (MS) and shown in FIG. 2B. To generate FIG. 2C, L929-mRuby-hIRF3 cells transduced with indicated shRNAs were stimulated with VACV70 (2 μg/ml) for 2 h or 4 h. Phosphorylation of TBK1 and IRF3 were estimated by Western blotting. To generate FIG. 2D, protein levels were assessed in cell lysates from L929-mRuby-hIRF3 cells transduced with indicated shRNAs by Western blotting. To generate FIG. 2E, L929-mRuby-hIRF3 cells transduced with indicated shRNAs were stimulated with VACV70 (2 μg/ml) for 2 h or 4 h. LC3A conversion and degradation of p62, cGAS, and STING were estimated by Western blotting. L929-mRuby-hIRF3 cells transduced with lentiviral shDAPK3 or shCtrl were treated with MG132 (20 μM) or Lactacystin (5 μM) for 6 h. Expression level of STING and LC3A conversion were assessed by Western blotting. L929-mRuby-hIRF3 cells transduced with lentiviral shDAPK3 or shCtrl were treated with Bafilomycin (100 μM) for 4 h or E64d (25 μM)+Pepstatin A (50 μM) for 8 h. Expression level of STING and LC3A conversion were assessed by Western blotting. To generate FIG. 2F, HEK293T cells were co-transfected with expression plasmids encoding Flag-STING, HA-Ubiquitin in the presence or absence of V5-DAPK3-WT, D161A, or T180A mutant. 24 h after transfection, cells were treated with MG132 (20 μM) for 2 h, followed by immunoprecipitation with anti-Flag antibody. Protein expression were assessed by Western blotting. HEK293T cells were co-transfected with expression plasmids encoding Flag-STING, HA-Ubiquitin mutant in the presence or absence of V5-DAPK3-WT. 24 h after transfection, cells were treated with MG132 (20 μM) for 2 h, followed by immunoprecipitation with anti-Flag antibody. Protein expression were assessed by Western blotting.

FIGS. 3A-3D show the results of a global phosphoproteomic approach, revealing that DAPK3 regulates phosphorylation of DNA-activated proteins. FIG. 3A is a heat map of selected phosphorylated proteins that shows higher phosphorylation in shCtrl cells than shDAPK3 cells upon dsDNA stimulation. FIG. 3B is a heat map of ubiquitin-related enzymes scored in the analysis. To generate FIG. 3C, HEK293T cells were co-transfected with expression plasmids encoding V5-tagged DAPK3 and Flag-tagged TBK1-WT or TBK1-K38M mutant. At 24 h after transfection, cells were lysed and immunoprecipitation was performed with anti-Flag antibody. FIG. C shows endogenous proteins that interact with TBK1 scored in targeting MS. To generate FIG. 3D, L929-mRuby-hIRF3 cells were transduced with siCYLD, siBRUCE, or siCtrl. At 72 h post-transfection, cells were stimulated with poly dA:dT (1 μg/ml), VACV70 (2 μg/ml), or poly I:C (0.5 μg/ml) for 3 h. The percentage of IRF nuclear translocation was calculated from values for cells transduced with control siRNA. Cells were stimulated with VACV70 (2 mg/ml) for 2 h, or 4 h. Phosphorylation of TBK1 and IRF3 and STING degradation were assessed by Western blotting. L929-mRuby-hIRF3 cells were transduced with two independent shDAPK3, or two independent shTBK1 and stimulated with VACV70 (2 μg/ml) for 2 h or 4 h. Phosphorylation of CYLD(S418) were assessed by Western blotting.

FIG. 4 shows the interaction between DAPK3, STING, TBK1, and various proteins scored in phosphoproteomics.) HEK293T cells were co-transfected with expression plasmids encoding V5-tagged DAPK3, HA-tagged STING, and Flag-tagged TBK1 (WT or K38M mutant)—these results are shown in FIG. 4. L929 cells were stably transduced with Flag-tagged STING, and stimulated with VACV70 for 2 h or 4 h. Immunoprecipitaion was performed with anti-Flag antibody and protein interaction was examined by Western blotting. L929 cells were transduced with lentiviral shDAPK3 and subsequently transduced with V5-tagged DAPK3. Confocal microscopy were performed to examine localization of DAPK3 (V5), pTBK1, LC3, p62, and ATG9A (autophagy marker), Sec16b (trafficking protein), CPLD, BRUCE.

FIGS. 5A-5B show DAPK3 depletion in tumor cells increases in vivo tumor growth. To generate FIG. 5A, B16F10-OVA cells, LLC-RFP cells, and MC38 cells were stimulated with 2′3′-cGAMP and 3′3′-cGAMP (5 μg/ml for each) and Ifnb1 mRNA level was quantified by qPCR. To generate FIG. 5B, B16F10-OVA cells were transduced with two independent lentiviral shDAPK3 or shSTING, and then stimulated with 2′3′-cGAMP and 3′3′-cGAMP (5 μg/ml for each). Ifnb1 mRNA level was quantified by qPCR. DAPK3-, or STING-depleted B16F10-OVA cells were subcutaneously injected into both the flanks of STING-gt/gt mice. At 5 day after injection, 3′3′-cGAMP along with Lipofectamine 2000 were injected into the right flank and lipofectamine 2000 only into the left flank. Tumor size was measured by digital caliper every 2 days.

FIGS. 6A-6C show the results of a variety of experiments. To generate FIG. 6A, primary HUVECs were transfected with siDAPK3, siSTING, or non-targeting control siRNA (siCtrl). 72 h later, mRNA levels (left panel) were examined by qPCR and protein levels (right panel) were estimated by Western blotting. To generate FIG. 6B, immortalized HUVECs were transduced with Cas9-expressing lentivirus containing scramble sgRNA (control) or sgRNA^{DAPK3} targeting the complementary strand of the exon 2 of the DAPK3 gene (DAPK3^{KO}) (sequence shown in the black box). Single cell clones were selected by limiting dilution for each group and DNA near the sgRNA-targeting site was amplified by PCR and sequenced. The predicted location of the Cas9-induced double strand break (DSB) is annotated by a black arrowhead. Chromatograms show a 11-bp deletion (indicated by the black box and dotted line) compared to the control (left panel). Cells were stimulated with poly dA:dT (0.5 μg/ml), VACV70 (2 μg/ml), or poly I:C (1 μg/ml) for 3 h and nuclear IRF3 translocation were examined by immunofluorescence. Percentage of IRF3 translocation was normalized with control for each stimulation. To generate FIG. 6C, DAPK3^{KO} immortalized HUVECs were transfected with siDAPK1 or siCtrl for 72 h, and nuclear IRF3 translocation was examined by immunofluorescence. Percentage of IRF3 translocation was normalized with siCtrl for each stimulation.

FIGS. 7A-7G show the results of a variety of experiments. To generate FIG. 7A, L929-mRuby-hIRF3 cells were stimulated with poly (dA:dT) (1 ug/ml) for 3 h, and IRF3 translocation was quantified by immunofluorescence microscopy. Cells were counterstained with DAPI. To generate FIGS. 7B-7D, L929-mRuby-hIRF3 cells were transduced with 5 different lentiviral shDAPK3 or shControl. To generate FIG. 7B, cells were stimulated with poly dA:dT (1 ug/ml), VACV70 (2 ug/ml), and poly I:C (0.5 ug/ml) for 3 h to quantify IRF3 nuclear translocation. To generate FIG. 7C, mRNA level of DAPK3 and STING were quantified by RT-qPCR. To generate FIG. 7D, Correlation of DAPK3 mRNA level and VACV70-induced IRF3 nuclear transloation. To generate FIGS. 7E-7F, L929-mRuby-hIRF3 cells were transfected with siDAPK3 (#1 and #4), sicGAS, siSTING, or siControl. 72 h later, for FIG. 7E, cells were stimulated with AF647-labelled poly (dA:dT) (1 ug/ml) for 1.5 h, and DNA uptake was examined by flow cytometric analysis and, in FIG. 7F, indicated protein expression was examine by immunoblot analysis. To generate FIG. 7G, HEK293T-cGAS-Clover cells were transduced with lentiviral shDAPK3 or shControl. Cells were stimulated with poly dA:dT (0.5 ug/ml) for 12 h, and intracellular cGAMP levels in cell lysate were measured by mass spectrometry.

FIGS. 8A-8E show the results of a variety of experiments. To generate FIG. 8A, primary HUVECs were transfected with 4 individual siDAPK3 or siSTING, were analyzed by Western blotting (left panel). Each protein expression level were normalized with β-actin and the ratio was quantified using Image J (right panel). To generate FIG. 8B. THP-ISG cells were transduced with two individual lentiviral shDAPK3 or shSTING and protein levels indicated were estimated by Western blotting. To generate FIG. 8C, primary HUVECs were transduced with lentiviral expression plasmids encoding V5-tagged luciferase, V5-tagged DAPK3-D161A mutant, or V5-tagged DAPK3-T180A mutant. 24 h later, cells were stimulated with poly dA:dT (0.5 μg/ml) or poly I:C (1 μg/ml) for 3 h, and the percentage of V5-positive cells containing nuclear IRF3 was examined by immunostaining. To generate FIG. 8D, L929-mRuby-hIRF3 cells were transduced with lentiviral shDAPK3, followed by lentiviral transduction of V5-tagged luciferase, DAPK3-WT, DAPK3-D161A, or DAPK3-T180A. Cells were stimulated with poly dA:dT (1 μg/ml) or poly I:C (0.5 μg/ml) for 2 h, and nuclear IRF3 translocation was examined by immunofluorescence (left panel). Protein levels indicated were estimated by Western blotting (right Panel). FIG. 8E reports the number of phosphorylation sites detected in SILAC-labeled shCtrl cells and shDAPK3 cells in phosphoproteomics and principal component analysis of the normalized protein phosphorylation profiles of the indicated cells. FIG. 8E includes list of endogenous proteins immunoprecipitated with Flag-tagged TBK1 in HEK293T lysates transfected with expression plasmids encoding Flag-tagged TBK1 and V5-tagged DAPK3-D161A.

FIGS. 9A-9D show that DAPK3 is required for DNA-stimulated transcription factor activation and ISG induction. To generate FIG. 9A, L929-mRuby-hIRF3 cells transduced with shDAPK3 (#1 and #2) or shControl were treated with proteasome inhibitors (left panel) or autophagy inhibitors (right panel) and STING protein level was estimated by immunoblot analysis. FIG. 9B shows the results of immunoprecipitation and immunoblot analysis of HEK293T cells transfected with plasmids encoding HA-ubiquitin, Flag-STING, DAPK3-V5 (wild type, D161A, or T180A mutant). Cells were treated with MG132 (20 uM) for 4 h before lysis. FIG. 9C-9D show the results of immunoprecipitation and immunoblot analysis of HEK293T cells transfected with plasmids encoding each indicated HA-ubiquitin mutant, Flag-STING, DAPK3-V5 (WT). Cells were treated with MG132 (20 uM) for 4 h before lysis.

FIGS. 10A-10C show the results of a variety of experiments. FIG. 10A shows the results of immunoprecipitation (top) and immunoblot analysis (below) of HEK293T cells transfected with plasmids encoding DAPK3 wild type (WT)-V5 and TBK1 wild type (WT)-Flag or TBK1 kinase-dead mutant (K38M)-Flag to examine interaction between DAPK3 and TBK1. Also performed were immunoblot analysis of phosphorylated and non-phosphorylated DAPK3 in immunoprecipitates precipitated with anti-Flag antibody from whole cell lysates of HEK293T cells transfected for 24 h with plasmids encoding TBK1 (WT)-Flag and DAPK3 (D161A)-V5 and treated with phosphatase (+) or not (−). FIG. 10B shows the results of mass spectrometry to identify DAPK3 phosphorylation sites regulated by TBK1 using immunoprecipitates precipitated with anti-Flag antibody from whole cell lysates of HEK293T cells transfected for 24 h with plasmids encoding DAPK3 (D161A)-V5 and TBK1 (WT)-Flag, which was treated with BX795 or DMSO for 4 h, or DAPK3 (D161A)-V5 and TBK1(K38M)-Flag. Also performed were immunoblot analysis of HEK293T cells transfected with plasmids encoding indicated DAPK3 mutant-V5 and TBK1 (WT)-Flag or TBK1 (K38M)-Flag. In addition, immunoblot analysis (below) and immunoprecipitation analysis of HEK293T cells transfected for 24 h with plasmids encoding indicated DAPK3 mutant-V5 and MLC2-HA, and immunoprecipitation was performed with anti-HA antibody were performed. FIG. 10C shows the results of immunoprecipitation (top) and immunoblot analysis (below) of HEK293T cells transfected with plasmids encoding DAPK3 wild type (WT)-V5, STING-HA and TBK1 wild type (WT)-Flag or TBK1 kinase-dead mutant (K38M)-Flag to examine interaction between DAPK3 and STING.

FIGS. 11A-11D show the results of a variety of experiments. To generate FIG. 11A, primary HUVECs were transduced with lentiviral luciferase, kinase-dead DAPK3 D161A mutant, or kinase-decreased DAPK3 T180A mutant. Cells were stimulated with poly (dA:dT) (0.5 ug/ml) or poly I:C (1 ug/ml) for 3 h, and the percentage of V5 tag-positive cells containing nuclear IRF3 was examined by immunostaining. To generate FIG. 11B, L929-mRuby-hIRF3 cells transduced with lentiviral shDAPK3 were subsequently transduced with lentiviral luciferase, DAPK3 wild type (WT), kinase-dead DAPK3 D161A mutant (D161A), or kinase-decreased DAPK3 T180A mutant (T180A). Cells were stimulated with poly (dA:dT) (0.5 ug/ml) or poly I:C (1 ug/ml) for 3 h, and cells containing nuclear IRF3 was examined by immunostaining. FIG. 11C shows the results of a Western blot analysis of THP1-ISG cells transduced with lentiviral shDAPK3 (#1 and #2), shSTING (#1 and #2), or shControl. To generate FIG. 11D, THP1-ISG cells or L929-mRuby-hIRF3 cells (not shown) were pretreated with DAPK inhibitors (#1 and #2) for 1 h, and then stimulated with VACV70 (2 ug/ml) for 4 h. mRNA level of IFNb was quantified by RT-qPCR.

FIGS. 12A-12E show phosphoproteomic analysis identifies novel candidate substrates of DNA-activated kinases including DAPK3 and TBK1. FIG. 12A is a schematic representation of SILAC-based phosphoproteomics.L929-mRuby-hIRF3 cells were cultured with DMEM containing “light” lysine/arginine or “heavy” lysine arginine. Cells were subsequently transduced with lentiviral shDAPK3 or shControl, and light-labeled cells were mock-stimulated and heavy-labeled cells were stimulated with poly (dA:dT) for 2 h. Lysates were mixed together to perform mass spec analysis. FIG. 12B is a heat map and hierarchal clustering based on the SILAC ratio (log 2) of identified phosphosites. The 3 clusters discriminate between upregulated in shControl more than in shDAPK3 (cluster 1), comparably upregulated in shControl- and shDAPK3 (cluster 2), and not upregulated in either shControl or shDAPK3 upon poly (dA:dT) stimulation. An arrow indicates cluster 1. FIG. 12C shows the results of Principal Component analysis (PCA) of shControl and shDAPK3. FIG. 12D shows the results of Gene Ontology (GO) enrichment analysis of the cluster 1. FIG. 12E depicts the overlap of scored proteins in phosphoproteomics and TBK1(WT)-Flag interacting proteins identified from the lysates used in FIG. 10B.

FIGS. 13A-13C show the results of a variety of experiments. To generate FIG. 13A, MCA205 cells (3×10⁵ cells) transduced with lentiviral shDAPK3, shSTING, or shControl were subcutaneously injected into wild type C57BL6/J mice. Tumor volume was measured with digital caliper every three days after injection. FIG. 13B shows the results of flow cytometric analysis of tumor-infiltrating leukocytes isolated from MCA205 primary tumor. To generate FIG. 13C, MCA205 transduced with lentiviral shDAPK3 were subsequently transduced with lentiviral DAPK3 wild type (WT) or kinase dead DAPK3 mutant D161A. Cells (5×10⁵ cells) were subcutaneously injected into wild type C57BL6/J mice. Tumor volume was measured with digital caliper every three days after injection.

FIGS. 14A-14C show the results of a variety of experiments. To generate FIGS. 14A-14B, primary HUVECs (a) and Bone-marrow-derived macrophages (BMDMs) were transfected with siDAPK1, siDAPK3, siSTING, or siControl. 72 h later, cells were stimulated with poly (dA:dT) (0.1 ug/ml), poly I:C (0.1 ug/ml), or 2′3′-cGAMP (10 ug/ml) for 4 h. mRNA level of IFNb were quantified by RT-qPCR. To generate FIG. 14C, THP1-ISG cells transduced with each indicated lentiviral shRNA were infected with HSV1, MVA, VSV, and SeV for 4 h. mRNA level of IFNb were quantified by RT-qPCR.

FIGS. 15A-15D show the results of a variety of experiments. To generate FIG. 15A, B16F10 cells (1×10⁶ cells) transduced with indicated shRNAs were subcutaneously injected into wild type C57BL6/J mice. Tumor volume was measured with digital caliper every three days after injection. FIG. 15B shows the results of in vitro growth of MCA205 transduced with indicated shRNAs measured by Cell Titer Blue assay. FIG. 15C-15D shows the results of Western blot analysis of MCA205 (FIG. 15C) or B16F10-OVA (FIG. 15D) transduced with indicated shRNAs (left panels) and RT-qPCR of IFNb induced by STING agonists.

FIGS. 16A-16F show that DAPK3 regulates cytosolic DNA sensing via STING. To generate FIG. 16A, L929-mRuby-hIRF3 cells transfected with each siRNA were stimulated with 2′3′-cGAMP or DMXAA to examine the expression level of IFNb mRNA (left panel). Cells were stimulated with poly (dA:dT) (0.5 ug/ml) for 12 h to examine intracellular 2′3′-cGAMP level by mass spec (right panel). To generate FIG. 16B, THP1-ISG transduced with each shRNA were stimulated with 2′3-cGAMP, 3′3′-cGAMP, or DMXAA to examine the expression level of IFNb mRNA. FIG. 16C shows the results of an immunoblot analysis of VACV70-induced pTBK1 (S172), TBK1, pIRF3 (S396), and IRF3 in L929-mRuby-hIRF3 cells transduced with each shRNA. FIGS. 16D-16E shows the results of immunoblot analyses of L929-mRuby-hIRF3 cells (FIG. 16D) or THP1-ISG (FIG. 16E) transduced with each shRNA. FIG. 16F shows the results of immunoblot analysis of STING ubiquitination in HEK293T cells transfected with lentiviral DAPK3(WT), kinase-dead DAPK3 (D161A), or kinase-decreasing DAPK3 (T180A).

### DETAILED DESCRIPTION

It is to be understood that the present disclosure is not limited to particular aspects described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. In some aspects, the full bibliographic citation for which can be found immediately preceding the claims. Nothing herein is to be construed as an admission that the present technology is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

### Definitions

As used herein, the singular forms “a”, “an”, and “the” include plural referents unless the context clearly dictates otherwise. Thus, for example, reference to “an agonist” includes a plurality of agonists, including mixtures thereof similarly, reference to “an antagonist” includes a plurality of antagonists, including mixtures thereof. The term “at least one” intends one or more.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It also is to be understood, although not always explicitly stated, that the reagents described herein are merely exemplary and that equivalents of such are known in the art.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

As used herein, the term “administer” and “administering” intend introducing the therapeutic agent (e.g., polynucleotide, vector, cell, modified cell, population) into a subject. The therapeutic administration of this substance serves to modulate, attenuate any symptom, or prevent additional symptoms from arising. In one aspect, administration is provided to prevent or reduce the likelihood of developing a tumor or cancer, metastasis or recurrence, and the substance can be provided in advance of any visible or detectable symptom or after thereof. The therapy can be combined with other known anti-cancer therapies and can be administered as a first-line, second-line, third-line, fourth line, or fifth-line therapy. Routes of administration include, but are not limited to, intravenous, by infusion, oral (such as a tablet, capsule or suspension), topical, transdermal, intranasal, vaginal, rectal, subcutaneous intravenous, intra-arterial, intramuscular, intraosseous, intraperitoneal, epidural and intrathecal. Administration can be systemic or specific, for example specific to particular cells or a population thereof. In some embodiments, the cells or tissue receiving the therapy can comprise one or more endothelial cells, monocytes, or fibroblasts. Specific administration can be achieved through a variety of means depending on the agent. For example, small molecules may be formulated as prodrugs to only be activated by enzymes or processes unique to a particular cell type; similarly, polynucleotides can comprise targeting domains or promoters specific to certain cell types.

As used herein, the term “agonist” as it relates to the interaction between DAPK and STING pathway refers to an agent that is able to activate or enhance this interaction, such as but not limited to a protein, a peptide, a small molecule, an antibody, a bispecific antibody, an antibody derivative, a ligand mimetic, a nucleic acid, or a pharmaceutical composition. For example, an agonist can be an agent that upregulates DAPK expression, such as a vector comprising a polynucleotide encoding DAPK, optionally operatively linked to a regulator element such as a promoter. Other examples include an agonist of the interaction between DAPK and TBK1 and/or STING.

As used herein, the term “antagonist” as it relates to the interaction between DAPK and STING pathway refers to an agent that is able to inhibit or decrease this interaction, such as but not limited to a protein, a peptide, a small molecule, an antibody, a bispecific antibody, an antibody derivative, a ligand mimetic, a nucleic acid, or a pharmaceutical composition. For example, an agonist can be anti-DAPK1 antibody, such as those commercially available from Biocompare (see biocompare.com/pfu/110447/soids/479947/Antibodies/DAPK). Other examples of antagonists are agents that downregulate, inhibit, or knock out DAPK expression—such as a CRISPR/Cas mediated gene editing system with a guide RNA targeted to DAPK or an inhibitory nucleic acid (e.g., siRNA or shRNA). Additional examples include small molecules known to function as DAPK inhibitors are HS56 (CAS No. 922050-57-5), H148 (CAS No. 1892595-16-2), HS94 (CAS No. 1892594-93-2), HS-38 (CAS No. 1030203-81-6), or TC-DAPK 6 (CAS No. 315694-89-4), depicted in order below (available through BioCompare, Probe Chem, SigmaAldrich and/or other known vendors based on a CAS number search):

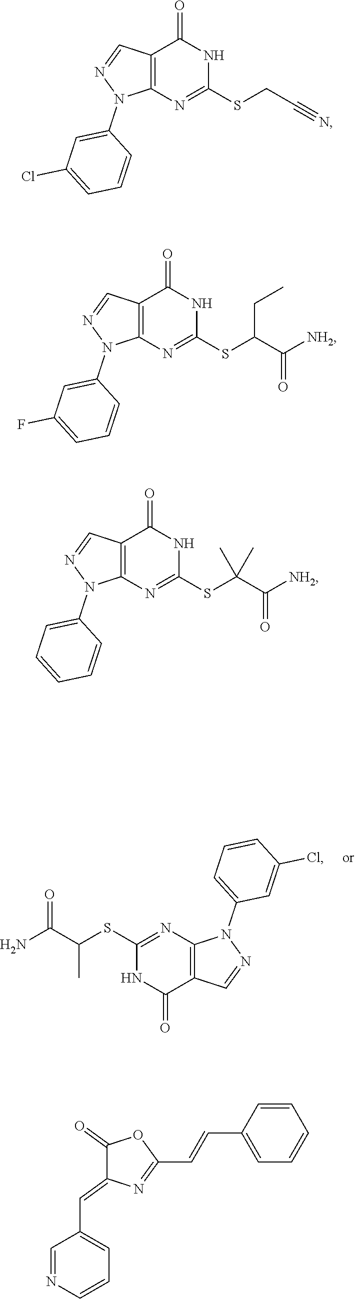 [SMILES: N#CCSc1nc2c(cnn2-c2cccc(Cl)c2)c(=O)[nH]1 ; CCC(Sc1nc2c(cnn2-c2cccc(F)c2)c(=O)[nH]1)C(N)=O ; CC(C)(Sc1nc2c(cnn2-c2ccccc2)c(=O)[nH]1)C(N)=O ; CC(Sc1nc2c(cnn2-c2cccc(Cl)c2)c(=O)[nH]1)C(N)=O ; O=C1OC(/C=C/c2ccccc2)=NC/1=C\c1cccnc1]

and pharmaceutically acceptable salts and solvates thereof. Further examples include an antagonist of the interaction between DAPK and TBK1 and/or STING.

As used herein, the term “subject” intends an animal, which in turn refers to living multi-cellular vertebrate organisms, a category that includes, for example, mammals and birds. The term “mammal” includes both human and non-human mammals. The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human. In some aspects, the subject is suffering from a disease or condition to be treated by one of the methods disclosed herein. In some aspects, the disease or condition is a viral infection or cancer, such as melanoma or colon cancer, e.g., colon carcinoma. In some aspects, the disease or condition is an inflammatory or autoimmune disease, such as polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility.

As used herein, the term “comprising” or “comprises” is intended to mean that the compositions and methods that include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating or preventing cancer or tumor. “Consisting of” shall mean excluding more than trace elements of other ingredients and substantial method steps. Embodiments defined by each of these transition terms are within the scope of this disclosure.

As used herein “DAPK” describes a Death-Associate Protein-Kinase and a gene or transcript encoding and expressing DAPK. Death-Associated Protein Kinases (DAPKs) are serine/threonine kinases that belong to the calmodulin (CaM)-regulated kinase superfamily. The family consists of three kinases, DAPK1 (also known as DAP-kinase, UniProt Ref. No. P53355), DAPK2 (also known as DRP-1, UniProt Ref. No. Q9UIK4), and DAPK3 (also known as ZIPK, UniProt Ref. No. 043293). All the family members work as regulators of cellular apoptosis and autophagy (Inbal (1997); Geering (2015); Kawai (1998); Bialik (2006), and they show a high degree of homology in their N-terminal kinase domain, suggesting that they share common functions and substrates. Accumulated studies have shown that DAPKs are important tumor suppressors (Gozuacik (2006); Raveh (2001). Hyper-methylation of DAPK1 and DAPK2 promoters that cause epigenetic silencing of DAPK was found in a variety types of cancer including chronic lymphocytic leukemia (CLL), non-small-cell lung carcinoma (NSCLC), and colorectal cancer (Katzenellenbogen (1999); Tang (2006); Mittag (2006)). In addition, loss-of-function mutations in DAPK3 isolated from primary human tumors promote cell proliferation and chemotherapeutic resistance in transformed cells (Brognard (2011). DAPK family members also have been shown to act as regulators of the innate immune responses (Lai (2013); Usui (2013). Recent reports demonstrated that DAPK1 interacts with IRF3 and IRF7 (Zhang (2014), and works as a negative feedback regulator of RIG-I pathway by direct phosphorylation of RIG-I (Willemsen (2017)), showing the link between the function of DAPK family and nucleic acid sensing pathway.

An “effective amount” or “efficacious amount” is an amount sufficient to achieve the intended purpose, non-limiting examples of such include: enhancement or downregulation of an innate immune response or increasing or decreasing type I interferon (IFN-I) expression. In one aspect, the effective amount is one that functions to achieve a stated therapeutic purpose, e.g., a therapeutically effective amount. As described herein in detail, the effective amount, or dosage, depends on the purpose and the composition, and can be determined according to the present disclosure by the treating physician or veterinarian.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

The terms “upregulate” and “downregulate” and variations thereof when used in context of gene expression, respectively, refer to the increase and decrease of gene, mRNA, or protein expression relative to a normal or expected threshold expression for cells, in general, or the sub-type of cell, in particular. Methods to determine expression level are known in the art and include sub-clinical and clinical methods, several of which are described herein.

As used herein the term “inhibit” means to silence gene, mRNA, or protein expression; nucleotides that accomplish this end include siRNA and shRNA.

The terms “knock out” and “knock in” refer to techniques of removing a gene or inserting a gene, respectively—such as but not limited to through viral-mediated editing, cre/lox, or CRISPR/Cas based systems.

The term “vector” can refer to any polynucleotide comprising an agent for delivery into a cell or system, wherein the agent is subsequently expressed in the cell or system—non-limiting examples include plasmids and viral constructs.

As used herein, the term “type I interferons” (IFN-I) refers to a subgroup of interferons that bind to IFN-α receptor (IFNAR) that consists of two chains (IFNAR1 and IFNAR2), including IFN-α, -β, -κ, -τ, -δ, -ξ, -ω, -v. IFN-Is have long been used as exogenous pharmaceuticals in cancer therapeutics targeting various types of cancer including chronic myeloid leukemia (CML) and melanoma (Zitvogel (2015). However, it is becoming increasingly appreciated that their pleiotropic immune modulatory effects on host immune cells are likely considerably more important than any anti-proliferative effects on the tumor cells themselves, most of which evolve defects in IFN-I signaling (Diamond (2011); Fuertes (2011); Dunn (2005); Swann (2007); Burnette (2011)). It was suggested that tumor-infiltrating CD11c⁺ cells were the main producers of IFN-I in tumor microenvironment by uptake of tumor cell-derived DNA (Deng (2014); Woo (2014) and/or mitochondrial DNA (Xu (2017)), which triggers cGAS-STING pathway activation, followed by the induction of T cell-dependent anti-tumor immune responses. However, dendritic cell-specific STING-deficient mice showed comparable subcutaneous tumor growth in the absence of CD47-SIRPa blockade, which stimulates phagocytosis of tumor cells (Xu (2017)), suggesting unknown mechanisms in natural immunosurveillance mediated by STING signaling. Other cell types including tumor endothelial cells (Demaria, 2015) and tumor cells themselves (Bidwell (2012); Sistigu (2014); Andzinski (2016) also can produce IFN-I. IFNβ mRNA level in MCA205-derived subcutaneous tumor was markedly downregulated in IFN β-deficient mice compared to wild type mice (Adzinski (2016)), and a chemotherapeutic reagent doxorubicin induces TLR3-dependent production of IFN-I and interferon stimulated genes (ISGs), which improves clinical responses to chemotherapy (Sitsigu (2014)). Other studies also showed that metastatic dissemination of human breast cancer into bones were closely associated with impaired production of IFN-I due to downregulation of IRF7 expression in tumor cells (Bidwell (2012)). In addition, decreased expression of STING has shown to be correlated with metastasis and poor prognosis in several human primary tumors (Xia (2016); Song (2017)), implicating the importance of STING signaling in tumor cells for tumor progression.

As used herein, the term “innate immune response” refers to a non-adaptive immune response, such as but not limited to recruitment of immune cells, activation of the complement cascade, identification and removal of foreign substances by specialized white blood cells, activation of the adaptive immune system through antigen presentation, and providing both physical and chemical barriers against foreign and/or infectious substances.

As used herein, the term “STING” relates to a protein that functions as a stimulator of interferon genes and the genes and transcript that express STING. STING (UniProt Ref. No. Q86WV6) is encoded by TMEM173. Also known as MITA, MPYS, and EMS, STING was identified as a transmembrane protein expressed in the endoplasmic reticulum (ER) (Ishikawa (2008); Jin (2008); Zhong (2008); Ishikawa (2009); Sun (2009)), and has been shown to be essential for production of numerous innate immune genes in response to immunostimulatory DNA and DNA/RNA viruses. Cells from STING-deficient mice showed significant defects in producing type I IFN (IFN-I) and other inflammatory cytokines induced by herpes simplex virus-1 (HSV-1) and Listeria monocytogenes infection as well as cell-free immunomodulatory dsDNA stimulation (Ishikawa (2008)). In addition, STING-deficient mice were shown to be more susceptible in HSV-1 infection and RNA virus infection including VSV (Ishikawa (2009)), Sendai virus (SeV), Influenza A virus (Holm (2016)).

Upon DNA sensing, a unique second messenger cyclic-GMP-AMP (“cGAMP”) is produced by cGAS (cGAMP synthase) in the presence of ATP and GTP, which activates STING (Sun (2012); Wu (2012); Ablasser (2013)). Then STING traffics from ER to Golgi apparatus, which in turn associates with TANK-binding kinase 1 (TBK1) (Sharma (2003); Fitzgerald (2003)), leading to the activation of a key innate transcription factor interferon regulatory factor 3 (IRF3).

The term “TBK1” refers to this crucial TANK-binding kinase 1 (UniProt Ref. No. Q9UHD2). Notably, recent studies confirmed that innate immune sensing of immunostimulatory DNA though the STING pathway is essential for eliciting immunity to a variety of immunogenic tumors (Woo (2015)). Moreover, the effects of a new cancer immunotherapy using monoclonal antibodies for blockade of PD-1/PD-L1 signaling (Pardoll (2012)) has been shown to largely depend on STING-mediated anti-tumor responses, which is boosted by injection of STING agonists, indicating that the STING pathway is a novel promising target for cancer immunotherapy (Corrales (2015); Fu (2015)).

As used herein, the term “interaction between DAPK3 and STING pathway” refers to the interaction between DAPK3 and one or more pathway components of the pathway involving STING activation and, optionally, IFN-1 secretion. Various pathway components are disclosed herein, such as but not limited to cGAMP, TBK1, and STING. It is further appreciated that DAPK3 can interact with one or more of these components directly or indirectly affect their activation. For example, using a HEK293T overexpression system, Applicants found that DAPK3 interacts with TBK1 and that DAPK3 interacts with STING in the presence of kinase active TBK1. The nature of this interaction is further characterized in the experiments disclosed herein. Applicants further found that DAPK3 regulates IFN-1 (specifically IFN-β) production induced by cGAMP stimulation, which specifically activates STING. This process is further characterized in the experiments disclosed herein. Not to be bound by theory, Applicants believe that DAPK3 interacts with the STING pathway along at least two avenues: (1) by affecting cGAMP stimulation along the STING pathway, thus, affecting the activation of STING and (2) by interacting with STING in the presence of kinase active TBK1.

As used herein, “treating” or “treatment” of a disease or condition in a subject refers to (1) inhibiting or reducing or preventing the symptoms or disease or condition from occurring in a subject that is predisposed or does not yet display symptoms of the disease; (2) inhibiting, reducing or preventing the disease or condition or arresting its development; or (3) ameliorating or causing regression of the disease or condition or the symptoms of the disease or condition. As understood in the art, “treatment” is an approach for obtaining beneficial or desired results, including clinical results. For the purposes of the present technology, beneficial or desired results can include one or more, but are not limited to, prevention, alleviation or amelioration of one or more symptoms, diminishment of extent of a condition (including a disease), stabilized (i.e., not worsening) state of a condition (including disease), prevention, delay or slowing of condition (including disease), progression, amelioration or palliation of the condition (including disease), states and remission (whether partial or total), whether detectable or undetectable. In one aspect, the term “treatment” excludes prevention.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

### MODES OF CARRYING OUT THE DISCLOSURE

Cell-intrinsic innate immunity constitutes the early essential and ubiquitous defensive response of susceptible cells to microbial and neoplastic pathogens. Though loss of function screening of tumor suppressor genes in primary human cells, Applicants newly identified death associated protein kinases (DAPKs) as regulators of the innate immune STING pathway, which was recently shown to be essential for mounting mature anti-tumor immunity in vivo. DAPK mutations found in human primary tumors have been shown to promote cancer cell survival and proliferation. The depletion of DAPK expression or loss of DAPK kinase activity in different primary and transformed cell types specifically impairs signaling though STING and downstream production of innate immune response genes, including the immunomodulatory type I interferon (IFN-I) and many other key inflammatory mediators. To identify substrates of DAPK, Applicants performed global phospho-proteomic profiling of DAPK deficient cells using SILAC-based mass spectrometry, demonstrating that DAPK controls inducible phosphorylation of an entire network of proteins involved in modulating the innate immune response.

### In General

Broadly, disclosed herein is a method of modulating an immune response, the method comprising, or alternatively consisting essentially thereof, or yet further consisting of, modulating the interaction between a DAPK and (STING) pathway by administering a modulator. In some embodiments, thea method comprises modulating an immune response in a subject. In certain embodiments, an immune response is an innate immune response. In some embodiments, a method comprises inhibiting, blocking or decreasing an immune response. In other embodiments, a method comprises increasing, enhancing, promoting or eliciting an immune response. In certain embodiments, a method comprises contacting a DAPK or STING with an agent that modulates the interaction between the DAPK and the STING pathway. In some embodiments, an agent is an antagonist of the interaction between the DAPK and the STING pathway. In certain other embodiments, an agent is an agonist of the interaction between the DAPK and the STING pathway. In some embodiments, a method comprises contacting a DAPK or TBK1 with an agent that modulates the interaction between the DAPK and TBK1. In certain embodiments, an agent is an antagonist of the interaction between the DAPK and TBK1. In certain alternative embodiments, an agent is an agonist of the interaction between the DAPK and TBK1. In some embodiments, an agent is a protein, peptide, small molecules, antibody, bispecific antibody, antibody derivative, ligand mimetic, nucleic acid or pharmaceutical composition. In certain embodiments, the DAPK is DAPK1, DAPK2, or DAPK3. In some embodiments, the subject is a mammal. In alternative embodiments, the subject is a human.

In some embodiments, the subject has a disease. In certain embodiments, the subject has an inflammatory or autoimmune disease. In some embodiments, the inflammatory or autoimmune disease is polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility. In some embodiments, a method comprises treating the subject for the inflammatory or autoimmune disease or a side-effect or symptom thereof.

Disclosed herein is a method of treating a subject for an inflammatory or autoimmune disease, or a side-effect or symptom thereof, wherein the method comprises administering an agent that modulates the interaction between a DAPK and the STING pathway. In some embodiments, an agent is an antagonist of the interaction between the DAPK and the STING pathway. In still other embodiments, an agent is an antagonist of the interaction between the DAPK and TBK1.

In some embodiments, the subject has cancer. In certain embodiments, a method comprises treating the subject for cancer or a side-effect or symptom.

Disclosed herein is a method of treating a subject for cancer or a side-effect or symptom thereof, the method comprising administering an agent that modulates the interaction between a DAPK and the STING pathway. In some embodiments, an agent is an agonist of the interaction between the DAPK and the STING pathway. In other embodiments, an agent is an agonist of the interaction between the DAPK and TBK1. In certain embodiments, an agent is a protein, peptide, small molecules, antibody, bispecific antibody, antibody derivative, ligand mimetic, nucleic acid or pharmaceutical composition.

Disclosed herein is a pharmaceutical composition comprising an agent that modulates the interaction between a DAPK and the STING pathway. In certain embodiments, an agent is an antagonist of the interaction between the DAPK and the STING pathway. In alternative embodiments, an agent is an agonist of the interaction between the DAPK and the STING pathway. In some embodiments, an agent is an antagonist of the interaction between the DAPK and TBK1. In alternative embodiments, an agent is an agonist of the interaction between the DAPK and TBK1. In certain embodiments, the DAPK is DAPK1, DAPK2, or DAPK3. In other embodiments, an agent is a protein, peptide, small molecules, antibody, bispecific antibody, antibody derivative, ligand mimetic or nucleic acid.

Methods of Enhancing an Innate Immune Response and/or Increasing Type I Interferon Expression

Aspects of the disclosure relate to a method of enhancing an innate immune response in a subject comprising, or consisting of, or alternatively consisting essentially of, administering an effective amount of an agonist of the interaction between DAPK and stimulator of interferon genes protein (STING) pathway. In some embodiments, the subject is a mammal, optionally a human. In some embodiments, the DAPK is DAPK1, DAPK2, or DAPK3. In some embodiments, the agonist is specifically administered to one or more of endothelial cells, monocytes, or fibroblasts.

Successful enhancement of the innate immune response includes increasing, enhancing, promoting or eliciting one or more aspects of the innate immune response. This can be readily determined by an increase in the baseline level of innate immune system pathway components, an increase of type I interferon expression or levels of cGAMP or STING (specific regulators of the cGAS-STING pathway). Alternatively, success may be determined by the resolution of one or more symptoms of a disease or disorder associated with the downregulation or inhibition of the immune response or an insufficient innate immune response, such as a viral infection or cancer. Such embodiments are disclosed below.

In some embodiments, the subject has a viral infection, optionally a CMV or Senai virus infection. Thus, in some embodiments, the viral infection can be treated by the administration of an effective amount of the agonist of the interaction between DAPK and stimulator of interferon genes protein (STING) pathway. Successful treatment of a viral infection can be determined by a variety of methods known in the art, such as but not limited to the determination of a reduction in one or more symptoms of the viral infection, a lack of a detectable viral genome in a biological sample from the subject (e.g., by PCR, qPCR, rtPCR, or Western blot) or an absence of biomarkers associated with viral infection.

In some embodiments, the subject has cancer, optionally melanoma or colon carcinoma. Thus, in some embodiments, the cancer can be treated by the administration of an effective amount the agonist of the interaction between DAPK and stimulator of interferon genes protein (STING) pathway. In some embodiments, the treatment may further comprise administration of one of more agents to blockade PD-1/PD-L1 signaling, such as but not limited to anti-PD-1 or PD-L1 antibodies, optionally monoclonal antibodies. Non-limitng examples of agents used in such a blockade include but are not limited to prembrolizumab (Merck), Nivolumab (Opdivo), pidilizumab (Cure Tech), AMP-224 (GSK), AMP-514 (GSK), PDR001 (Novartis), cemipilmab (Regeneron and Sanofi), Atezolizumab (Genentech), Avelumab (Merck Serono and Pfizer), Durvalumab (AstraZeneca), BMS-936559 (Bristol-Meyers Squibb), CK-301 (Checkpoint Therapeutics). In some embodiments, the agonist is specifically administered to a tumor cell. Successful treatment of cancer can be determined by a variety of methods known in the art, such as but not limited to the determination of a reduction in tumor size or tumor burden, a resolution of metastases, or a reduction of circulating tumor cells in a biological sample from the subject.

Further aspects relate to a method of increasing type I interferon (IFN-I) expression in a population of cells comprising, or consisting of, or alternatively consisting essentially of, administering an effective amount of an agonist of the interaction between DAPK and stimulator of interferon genes protein (STING) pathway to a population of cells. In some embodiments, the DAPK is DAPK1, DAPK2, or DAPK3. In some embodiments, the population of cells comprises, or consists of, or alternatively consists essentially of, one or more of endothelial cells, monocytes, or fibroblasts.

A successful increase in type I interferon expression can be determined by a number of methods known in the art, for example, detecting an increased level or type I interferon circulating in a biological sample (e.g., by PCR, qPCR, rtPCR, or Western blot) or any downstream STING pathway components from a baseline determined by conducting the same assay prior to treatment and/or a “normal” population value.

Both method aspects disclose in this section employ an agonist of the interaction between DAPK and stimulator of interferon genes protein (STING) pathway. In some embodiments, the agonist is a protein, peptide, small molecule, antibody, bispecific antibody, antibody derivative, ligand mimetic, nucleic acid, or pharmaceutical composition. In some embodiments, the agonist upregulates DAPK expression. In further embodiments, the agonist is a vector comprising a polynucleotide encoding DAPK, optionally operatively linked to a regulatory element. In some embodiments, the agonist is an agonist of the interaction between DAPK and TBK1 and/or STING.

Agonists can be readily generated using information known in the art, for example based on a protein library screen or computational models. A number of exemplary predictive algorithms can be utilized to predict the structure of agonists based on the identification of active sites through the generation of successful antagonists (e.g., those disclosed herein below), such as but not limited to ligand based, structure based, and hybrid models optionally applying molecular dynamics and/or quantum mechanics based approaches for agonist prediction. General screens are also accomplishable based on analogy to STING pathway components that interact with DAPK, such as TBK1.

Methods of Downregulating an Innate Immune Response and/or Decreasing Type I Interferon Expression

Aspects of the disclosure relate to a method of downregulating an innate immune response in a subject comprising, or consisting of, or alternatively consisting essentially of, administering an effective amount of an antagonist of the interaction between DAPK and stimulator of interferon genes protein (STING) pathway. In some embodiments, the subject is a mammal, optionally a human. In some embodiments, the DAPK is DAPK1, DAPK2, or DAPK3. In some embodiments, the antagonist is specifically administered to one or more of endothelial cells, monocytes, or fibroblasts.

Successful downregulation of the innate immune response includes inhibiting, blocking or decreasing one or more aspects of the innate immune response. This can be readily determined by a decrease in the baseline level of innate immune system pathway components, such as a decrease of type I interferon expression or levels of cGAMP or STING (specific regulators of the cGAS-STING pathway). Alternatively, success may be determined by the resolution of one or more symptoms of a disease or disorder associated with the upregulation of the immune response, such as an inflammatory or autoimmune disease. Such embodiments are disclosed below.

In some embodiments, the subject has an inflammatory or autoimmune disease, such as polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility. In some embodiments, a method comprises treating the subject for the inflammatory or autoimmune disease or a side-effect or symptom thereof. Thus, in some embodiments, the inflammatory or autoimmune disease can be treated by the administration of the antagonist of the interaction between DAPK and STING pathway. Successful treatment of a viral infection can be determined by a variety of methods known in the art, such s as but not limited to the determination of a reduction in one or more symptoms of the inflammatory or autoimmune disease or an absence of biomarkers associated with the inflammatory or autoimmune disease.

Further aspects relate to a method of decreasing type I interferon (IFN-I) expression in a population of cells comprising, or consisting of, or alternatively consisting essentially of, administering an effective amount of an antagonist of the interaction between DAPK and STING pathway to a population of cells. In some embodiments, the DAPK is DAPK1, DAPK2, or DAPK3. In some embodiments, the population of cells comprises, or consists of, or alternatively consists essentially of, one or more of endothelial cells, monocytes, or fibroblasts.

A successful decrease in type I interferon expression can be determined by a number of methods known in the art, for example, detecting a decreased level or type I interferon circulating in a biological sample (e.g., by PCR, qPCR, rtPCR, or Western blot) or any downstream STING pathway components from a baseline determined by conducting the same assay prior to treatment and/or a “normal” population value.

Both method aspects disclosed in this section administer an antagonist of the interaction between DAPK and STING pathway. In some embodiments, the antagonist is a protein, peptide, small molecule, antibody, bispecific antibody, antibody derivative, ligand mimetic, nucleic acid, or pharmaceutical composition. In some embodiments, the antagonist is an anti-DAPK1 antibody, such as those provided in the BioCompare catalog or generated through known methods against DAPK. In some embodiments, the antagonist is a small molecule, such as HS56 (CAS No. 922050-57-5), H148 (CAS No. 1892595-16-2), HS94 (CAS No. 1892594-93-2), HS-38 (CAS No. 1030203-81-6), or TC-DAPK 6 (CAS No. 315694-89-4), depicted in order below:

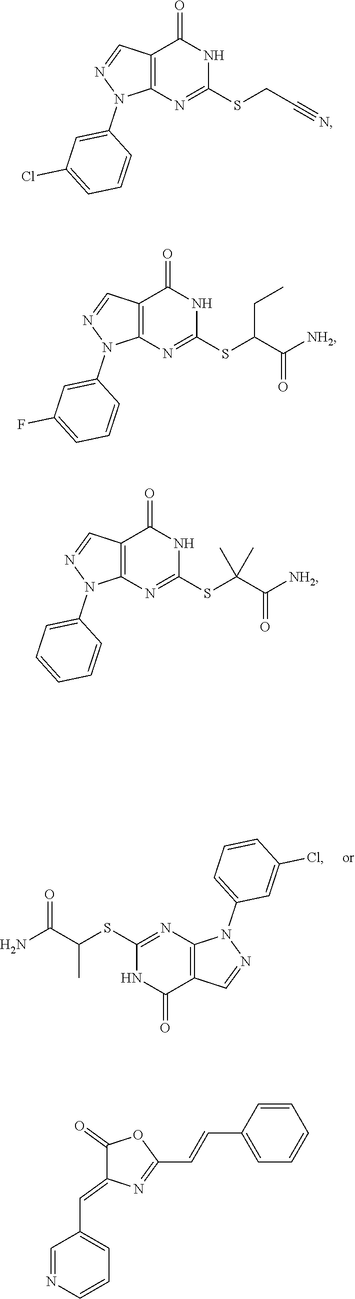 [SMILES: N#CCSc1nc2c(cnn2-c2cccc(Cl)c2)c(=O)[nH]1 ; CCC(Sc1nc2c(cnn2-c2cccc(F)c2)c(=O)[nH]1)C(N)=O ; CC(C)(Sc1nc2c(cnn2-c2ccccc2)c(=O)[nH]1)C(N)=O ; CC(Sc1nc2c(cnn2-c2cccc(Cl)c2)c(=O)[nH]1)C(N)=O ; O=C1OC(/C=C/c2ccccc2)=NC/1=C\c1cccnc1]

or a pharmaceutically acceptable salt or solvate thereof. In some embodiments, the antagonist downregulates, inhibits, or knocks out DAPK expression, non-limiting examples of such include for example an inhibitory nucleic acid, optionally an siRNA or shRNA, such as those directed against the DAPK targets provided in Table 1 below. In some embodiments, the antagonist is an antagonist of the interaction between DAPK and TBK1 and/or STING.

Agonists can be generated using information known in the art, for example based on a protein library screen or computational models. For example, a number of predictive algorithms can be utilized to predict the structure of antagonists utilizing one or more of the molecules noted above to screen for similar agents having similar structure and/or biological function, such as but not limited to ligand based, structure based, and hybrid models optionally applying molecular dynamics and/or quantum mechanics based approaches for agonist prediction. General screens are also accomplishable based on analogy to STING pathway components that interact with DAPK and result in its inactivation.

### Dosing, Formulations, and Routes of Administration

With respect to the agonists and antagonists disclosed herein, it is appreciated that dosing can be accomplished in accordance with the methods disclosed herein using capsules, tablets, oral suspension, suspension for intra-muscular injection, suspension for intravenous infusion, get or cream for topical application, or suspension for intra-articular injection.

Dosage, toxicity and therapeutic efficacy of the agonists and antagonists described herein can be determined by standard pharmaceutical procedures in cell cultures or experimental animals, for example, to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In certain embodiments, the agonists and antagonists exhibit high therapeutic indices. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies (in certain embodiments, within a range of circulating concentrations that include the ED50 with little or no toxicity. The dosage may vary within this range depending upon the dosage form employed and the route of administration utilized. For any agonist or antagonist used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, an effective amount of an agonist or antagonist disclosed herein sufficient for achieving a therapeutic or prophylactic effect ranges from about 0.000001 mg per kilogram body weight per administration to about 10,000 mg per kilogram body weight per administration. Suitably, the dosage ranges are from about 0.0001 mg per kilogram body weight per administration to about 100 mg per kilogram body weight per administration. Administration can be provided as an initial dose, followed by one or more “booster” doses. Booster doses can be provided a day, two days, three days, a week, two weeks, three weeks, one, two, three, six or twelve months after an initial dose. In some embodiments, a booster dose is administered after an evaluation of the subject's response to prior administrations.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The agonists and antagonists disclosed herein can be formulated as pharmaceutical compositions, as disclosed herein above, comprising an active agent and a carrier. The carriers can be one or more of a solid support or a pharmaceutically acceptable carrier. The compositions can further comprise an adjuvant or other components suitable for administrations as vaccines. In one aspect, the compositions are formulated with one or more pharmaceutically acceptable excipients, diluents, carriers and/or adjuvants. In addition, embodiments of the compositions of the present disclosure include one or more of the agonists or antagonists disclosed herein.

For oral preparations, one or more of the agonists or antagonists disclosed herein can be used alone or in pharmaceutical formulations disclosed herein comprising, or consisting essentially of, the active agent in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The disclosure provides pharmaceutical formulations in which the one or more of the agonists or antagonists disclosed herein can be formulated into preparations for injection in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. For intravenous administration, suitable carriers include physiological bacteriostatic water, Cremophor EL™ (BASF, Parsippany, N.J.), or phosphate buffered saline (PBS). In all cases, a composition for parenteral administration must be sterile and should be fluid to the extent that easy syringability exists.

Aerosol formulations provided by the disclosure can be administered via inhalation and can be propellant or non-propellant based. For example, embodiments of the pharmaceutical formulations disclosed herein comprise one or more of the agonists or antagonists disclosed herein formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself (e.g., by an elastic bladder)).

Suppositories disclosed herein can be prepared by mixing a compound disclosed herein with any of a variety of bases such as emulsifying bases or water-soluble bases. Embodiments of this pharmaceutical formulation of a compound disclosed herein can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds disclosed herein. Similarly, unit dosage forms for injection or intravenous administration may comprise a compound disclosed herein in a composition as a solution in sterile water, normal saline or another pharmaceutically acceptable carrier.

Embodiments of the pharmaceutical formulations disclosed herein include those in which one or more of the agonists or antagonists disclosed herein is formulated in an injectable composition. Injectable pharmaceutical formulations disclosed herein are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations disclosed herein.

In an embodiment, one or more of the agonists or antagonists disclosed herein is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound disclosed herein can be accomplished using any of a variety of refillable, pump systems. Pumps provide consistent, controlled release over time. In some embodiments, a compound disclosed herein is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electro osmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electro diffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems may be utilized due to their generally more consistent, controlled release over time. Osmotic pumps are used in some embodiments due to their combined advantages of more consistent controlled release and relatively small size (see, e.g., PCT International Pat. Application Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a composition disclosed herein are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylatanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the one or more of the agonists or antagonists disclosed herein is delivered in a controlled release system. For example, a composition disclosed herein may be administered using intravenous infusion, an implantable osmotic pump, a transdermal patch, liposomes, or other modes of administration. In one embodiment, a pump may be used (Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the liver, thus requiring only a fraction of the systemic dose. In yet another embodiment, a controlled release system is placed in proximity of the therapeutic target, thus requiring only a fraction of the systemic. Other controlled release systems are discussed in the review by Langer (1990) Science 249:1527-1533.

In another embodiment, the compositions of the present disclosure (as well as combination compositions separately or together) include those formed by impregnation of an inhibiting agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

The present disclosure provides methods and compositions relating to administration of the agonists and antagonists disclosed herein. In various embodiments, these methods disclosed herein span almost any available method and route suitable for drug delivery, including in vivo and ex vivo methods, as well as systemic and localized routes of administration.

Routes of administration applicable to the methods disclosed herein include intranasal, intramuscular, urethrally, intratracheal, subcutaneous, intradermal, topical application, intravenous, rectal, nasal, oral, inhalation, and other enteral and parenteral routes of administration. Routes of administration may be combined, if desired, or adjusted depending upon the agent and/or the desired effect. An agonist or antagonist can be administered in a single dose or in multiple doses. Embodiments of these methods and routes suitable for delivery, include systemic or localized routes. In general, routes of administration suitable for the methods disclosed herein include, but are not limited to, direct injection, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

The agonists and antagonists disclosed herein can also be delivered to the subject by enteral administration. Enteral routes of administration include, but are not limited to, oral and rectal (e.g., using a suppository) delivery.

Methods of administration of the active through the skin or mucosa include, but are not limited to, topical application of a suitable pharmaceutical preparation, transcutaneous transmission, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods disclosed herein, the agonist or antagonist will be administered by inhalation, injection or orally on a continuous, daily basis, at least once per day (QD), and in various embodiments two (BID), three (TID), or even four times a day.

### Kits

The present disclosure also provides kits for performing any of the methods disclosed herein as well as component and instructions for carrying out the methods of the present disclosure such as modulating the innate immune response and/or IFN-I expression.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components can be packaged in a manner customary for use by those of skill in the art. For example, these suggested kit components may be provided in solution or as a liquid dispersion or the like.

### Examples

The following examples are intended to illustrate, and not limit the scope of this disclosure.

The examples disclosed herein are the first to establish DAPK as master regulators of the STING-IFN-I pathway in cancer, and provide an important link between tumor suppressors and the development of cellular innate immunity.

### Materials and Methods

Antibodies and Reagents: Poly (dA:dT) and PMA (P8139) were purchased from Sigma. AF647-labeled poly dA:dT was purchased from IBA. Poly I:C (LMW) and Lyovec were obtained from Invivogen. VACV70 was ordered from IDT. Jetprime transfection reagent was obtained from VWR. Lipofectamine 2000 and mouse anti-V5 antibody (Ab) were obtained from Life technologies. Rabbit polyclonal anti-IRF3 Ab (sc-9082), rabbit polyclonal anti-NFκB p65 Ab (sc-372), anti-CPLD Ab (sc74435), mouse monoclonal anti-ubiquitin (P4D1) Ab (sc-8017) and horse radish peroxidase (HRP)-conjugated goat anti-guinea pig secondary antibody were obtained from Santa Cruz. Rabbit monoclonal anti-phospho-TBK1 (Ser172) Ab (5483S), rabbit monoclonal anti-TBK1 Ab (3504S), rabbit polyclonal anti-phospho-IRF3 (Ser396) Ab (4947S), rabbit polyclonal anti-DAPK1 Ab (3008S), rabbit monoclonal anti-cGAS Ab (31659S), rabbit monoclonal anti-STING (D2P2F) Ab (13647S), rabbit monoclonal anti-LC3A Ab (4599S), and RIPA buffer (10×) (9806S) were obtained from Cell Signaling Technology. Rabbit polyclonal anti-cGAS (AP10510c) and rabbit polyclonal anti-STING (AP9747b) were obtained from Abgent. Mouse monoclonal anti-TBK1/IKKc Ab (NB100-56524) was obtained from Novus Biologicals. Rabbit polyclonal anti-DAPK3 Ab (ab79422), rabbit polyclonal anti-MAVS Ab (ab31334), rabbit polyclonal anti-DDX58 Ab (ab45428), mouse monoclonal anti-HA (12CA5) Ab (ab16918) were obtained from Abcam. Rabbit polyclonal anti-DAPK3 (LS-B11575) was purchased from LifeSpan Biosciences. Polyclonal guinea pig anti-SQSTMl/p62 Ab was obtained from Progen Biotechnik. Mouse monoclonal anti-Flag (M2) Ab (F1804 and F3165), rabbit anti-Flag Ab (F7425), rat polyclonal anti-HA (3F10) Ab, rabbit anti-V5 antiserum (V8137), HRP-conjugated Goat anti-rabbit secondary antibody, HRP-conjugated Goat anti-mouse secondary antibody, HRP-conjugated rabbit anti-rat secondary antibody, and mouse monoclonal anti-β-actin Ab were obtained from Sigma. Alexa Fluor 647 AffiniPure goat anti-rabbit IgG(H+L) was obtained from Jackson Immunological Research.

Cells: Primary human umbilical vein endothelial cells (HUVECs) from a single donor were cultured in EGM2 Bulletkit growth media (LONZA) at 37 C with 5% CO2 and were used below 5 passages. Neonatal healthy dermal fibroblasts (NHDFs; Clontech), HEK293T, A549 (ATCC), HeLa (ATCC), LLC-RFP (a gift from Dr. Catherine C. Hedrick, LJI), and L929 were cultured in Dulbecco's modified Eagle's medium (DMEM) supplemented with 10% fetal bovine serum (FBS), L-glutamine (2 mM), Penicillin-Streptomycin (100 U/ml) and HEPES (10 mM). 5 μg/ml Insulin (Sigma-Aldrich) and 1 ng/ml bFGF (Sigma-Aldrich) were added to NHDF culture media. MC38 was a gift from Dr. Jeffrey Schlom, National Cancer Institute, and cultured in DMEM supplemented with 10% FBS, L-glutamine (2 mM), non-essential amino acids (NEAA) (0.1 mM) and sodium pyruvate (0.1 mM), and Gentamicin sulfate (50 μg/ml). Bone-marrow-derived macrophages (BMDM) were cultured in RPMI supplemented with 10% FBS, L-glutamine (2 mM), NEAA (0.1 mM), 2-mercaptoethanol (55 μM), and sodium pyruvate (0.1 mM) in the presence of L929 culture supernatants. THP1-ISG was obtained from Invivogen and cultured in RPMI supplemented with 10% FBS, 2-mercaptoethanol (55 μM), 10 μg/ml Blasticidin (Life technologies) and 100 μg/ml Zeocin (Invivogen).

For induction of innate immune responses against nucleic acids, HUVECs and L929-mRuby-IRF3 cells were replated at 1×10⁴ cells per well in 96-well plates for immunostaining, at 1×10⁵ cells per well in 12-well plates for RNA extraction, and at 5×10⁵ cells in 6-well plates for Western blotting and cGAMP detection one day before stimulation. THP1-ISG cells were differentiated using 0.5 μM PMA for 3 h and plated at 3×10⁵ cells per well in 12-well plates one day before stimulation (Hornung, (2009), full citation provided below under “References”). Nucleic acids were transfected using Lyovec (for HUVECs), Jetprime (for L929-mRuby-hIRF3 cells), or Lipofectamine 2000 (for THP1-ISG cells and B16F10-OVA cells) according to manufacturer's instruction. IRF3 nuclear translocation was examined at 3 h post-stimulation, and RNA extraction was performed at 4 h post-stimulation.

Virus preparation and infection: HCMV MOLD clinical strain was prepared as previously described (Lio (2016), full citation provided below under “References”). HSV-1 and Sendai virus were purchased from ATCC. VSV was a gift from Dr. Shane Crotty, LJI, and VSV viral stocks were prepared by infecting BHK cells at a MOI of 0.1 and letting the virus harvested for 16˜18 h post infection. To purify virus, virus supernatants were pelleted by ultracentrifugation at 120,000×g through a 20% sucrose cushion for 1.5 h, followed by resuspension of the viral pellet in DMEM supplemented with 5% FBS. VSV stock titer was determined by plaque assay using Vero cells. Cells were infected with VSV viral stock and incubated at 37 C for 1 h, followed by overlaying warmed 2× medium 199 (Life Technologies) supplemented with 10% FBS and 1% agarose. After 24 h incubation, agarose layer was removed and cells were fixed with 10% PFA and stained with 0.1% crystal violet solution containing 25% ethanol. Virus infection of HUVECs were performed as previously described (Lio (2016), full citation provided below under “References”).

Generation of reporter cells: L929-mRuby-hIRF3 cells were generated as follows: HEK293T cells were transfected with pLV-EF1a-mRuby-IRF3 and the virus supernatants were used for lentiviral transduction into L929 cells. After puromycin selection, single clones were obtained with limiting dilution and IRF3 nuclear translocation was examined by immunocytochemistry. 293T-cGAS-clover cells were generated by transducing HEK293T cells with pLV-EF1a-cGAS-Clover, followed by puromycin selection.

Cellular uptake of nucleic acids: DNA uptake was examined as previously described with slight modifications (Imanishi (2014), full citation provided below under “References”). Briefly, 1×10⁵ cells were stimulated with 0.5 μg/ml AF647-poly (dA:dT) (IDT, diluted 1:4 with unlabeled poly (dA:dT)) at 37 C for 90 min. Cells were trypsinized and washed once with PBS/0.1% BSA followed by an acidic wash containing 100 mM acetic acid and 150 mM NaCl (pH 2.7) for 1 min to remove unbound and cell surface-bound labeled poly dA:dT. Cells were washed twice with PBS/0.1% BSA and analyzed fluorescence by LSR Fortessa.

siRNA targets: The target sequences of the siRNA and shRNA used in the examples disclosed herein are provided below in Table 1.

TABLE 1
Target sequences of siRNA and shRNA

| Human | | Dharmacon catalog |
|---|---|---|
| siRNA | Target Sequence | number |
| DAPK1 #1 | GAAGAAAUGCCGUGAGAAA | D-004417-02 |
| DAPK1 #2 | GAACAGGUUUGGAAAUGAU | D-004417-04 |
| DAPK1 #3 | GAUCAAGCCUAAAGAUACA | D-004417-07 |
| DAPK1 #4 | GGAAACAAUCCGUUCGCUU | D-004417-21 |
| DAPK3 #1 | GAACAUUCCUGGAUUAAGG | D-004947-01 |
| DAPK3 #2 | CCACGCGUCUGAAGGAGUA | D-004947-02 |
| DAPK3 #3 | GAGCCAGGCCCGUAAGUUC | D-004947-03 |
| DAPK3 #4 | GAGGAGUACUUCAGCAACA | D-004947-04 |
| STING #1 | GCACCUGUGUCCUGGAGUA | D-024333-01 |
| STING #2 | GGUCAUAUUACAUCGGAUA | D-024333-02 |
| STING #3 | GCAUCAAGGAUCGGGUUUA | D-024333-03 |
| STING #4 | ACAUUCGCUUCCUGGAUAA | D-024333-04 |
| Mouse | | Dharmacon catalog |
| siRNA | Target Sequence | number |
| DAPK1 #1 | GGAAUCACCUGCAAGAAAU | D-040260-01 |
| DAPK1 #2 | GAAUCAAGCUCAAACUGUU | D-040260-02 |
| DAPK1 #3 | AAACGAGGCUCAAGGAUUG | D-040260-03 |
| DAPK1 #4 | GAAGGAAUCUCUGACUGAA | D-040260-04 |
| DAPK3 #1 | UCUCAGCAGUGAACUAUGA | D-044800-01 |
| DAPK3 #2 | CCACGCAGUUCCUCAAACA | D-044800-02 |
| DAPK3 #3 | GGGAGGAGAUCGAACGCGA | D-044800-03 |
| DAPK3 #4 | CCGAGAUCGUGAACUAUGA | D-044800-04 |
| cGAS #1 | GGAUUGAGCUACAAGAAUA | D-055608-01 |
| cGAS #2 | AGAAAUCUCUGUGGAUAUA | D-055608-02 |
| cGAS #3 | GAAGAUCCGCGUAGAAGGA | D-055608-03 |
| cGAS #4 | GCUAAGAAGCCGUCCGCGA | D-055608-04 |
| STING #1 | CGAAAUAACUGCCGCCUCA | D-055528-01 |
| STING #2 | CAAAUCACACUCUGAAGUA | D-055528-02 |
| STING #3 | AACAUUCGAUUCCGAGAUA | D-055528-03 |
| STING #4 | GCAUCAAGAAUCGGGUUUA | D-055528-04 |

| Human | |
|---|---|
| shRNA | Target Sequence |
| DAPK3 #1 | CGUUCACUACCUGCACUCUAA |
| DAPK3 #2 | CGUCUGAAGGAGUACACCAUC |
| STING #1 | GCAUGGUCAUAUUACAUCGGA |
| STING #2 | GUUUACAGCAACAGCAUCUAU |
| Mouse | |
| shRNA | Target Sequence |
| DAPK3 #1 | GCAGUGAACUAUGACUUUGAU |
| DAPK3 #2 | GCAUGACGUGUUCGAGAACAA |
| STING #1 | AUGAUUCUACUAUCGUCUUAU |
| STING #2 | CAACAUUCGAUUCCGAGAUAU |

siRNA transfection: siRNAs were transfected into HUVECs or L929-mRuby-IRF3 cells by using DharmaFECT 4 (GE Dharmacon) at a final concentration of 25 nM or 40 nM, respectively, according to the manufacturer's instructions. Briefly, HUVECs or L929-mRuby-hIRF3 cells were trypsinized and plated at 5×10⁵ cells or 2×10⁵ cells, respectively, per well into 6-well plates the day before transfection. Fresh medium was replaced 1 h before transfection. siRNA and DharmaFECT 4 were each diluted with Opti-MEM (Life Technologies) and incubated at room temperature for 5 min before mixing. Lipid-siRNA complexes were incubated for an additional 30 min and added dropwise to cells. Medium was replaced at 24 h post transfection. Cells were trypsinized and plated after 48 h (1×10⁴ cells per well in 96-well plates for immunostaining, 1×10⁵ cells per well in 12-well plates for RNA extraction), and stimulated with nucleic acids or infected with viruses after 72 h post-transfection.

Total RNA extraction and quantitative PCR: Total RNA from cells was extracted by using the Quick-RNA Miniprep Plus Kit (Zymoresearch) following manufacturer's instruction, and cDNA synthesis was performed with the qScript cDNA synthesis kit (Quanta). Quantitative reverse transcription-PCR (qRT-PCR) was performed with a CFX96 Touch Detection System (Bio-Rad), using Taqman Universal PCR master mix (Applied Biosystems) or FastStart SYBR green Master (Roche). The mRNA level of each gene was normalized to the level of HPRT (human) and 18S rRNA (mouse) for Taqman assay, and HPRT (human) and β-actin (mouse) for SYBR.

Plasmids: pDONR223-DAPK3 was a gift from William Hahn & David Root (Addgene plasmid #23436) (Johannessen (2010)). pLenti-CMV-Puro-LUC (w168-1) was a gift from Eriv Campeau (Addgene plasmid #17477) (Campeau (2009)) and luciferase was ligated into pLX304 with Gateway cloning (ThermoFisher Scientific). HA-CYLD-WT was a gift from Stephen Elledge (Addgene plasmid #15506) (Stegmeier (2007)). pEF-BOS-huTBK1-FlagHis and pEF-BOS-hu-TBK1-K38M-Flag-His were obtained from a vendor. Human DAPK1, DAPK2, STING and cGAS clones were obtained from Precision LentiORF™ Collection (GE Healthcare). Human DAPK3-D161A, DAPK3-T180A, DAPK3-KD (amino acids 1-256), DAPK3-LZ (amino acids 257-436) were amplified by PCR and ligated into pLX304 with Gateway cloning (ThermoFisher Scientific). pEF-neo-HA-ubiquitin is a gift from Dr. Yun-Cai Liu(LJI). HA-ubiquitin-K110, -K270, -K480, -K630, HA-ubiquitin-K11R, -K27R, -K48R, -K63R were generated via gBlocks (IDT) and cloned into pEF-neo. Lentiviral shRNA vectors were obtained from MISSION® shRNA Library (Sigma). shRNA and sgRNA sequence targets used in the experiments are listed in Table 1. Human STING C-terminus (aa149-379) was cloned into pGEX-4T-2 (a gift from Dr. Dirk Zajonc, III).

Packaging plasmids, psPAX2 (Addgene plasmid #12260) and pMD2.G (Addgene plasmid #12259), were gifts from Didier Trono, and they were mixed 3:1 for transfection in HEK293T cells

Lentiviral transduction: 293T cells (4×105 cells per well in 6-well plates) were transfected with 375 ng of each shRNA vector and 375 ng of packaging plasmids mixture. Medium was replaced into DMEM supplemented with 30% FBS without antibiotics at 24 h post-transfection. For some experiments, viral titer was examined by HIV Type I p24 Antigen ELISA 2.0 (Zeptometrix, #0801008). L929-mRuby-hIRF3 cells, B16F10-OVA cells, or THP1-ISG were replated at 2×10⁵ cells per well in 6-well plates the day before transduction. Cells were infected with lentiviral shRNA in the presence of polybrene (TR-1003-G, EMD Millipore) with spin infection at 2000 rpm for 30 min. Cells were subsequently selected with puromycin at a concentration of 4 μg/ml (for L929-mRuby-hIRF3 cells) or 1 μg/ml (for B16F10-OVA cells and THP1-ISG). Human telomerase reverse transcriptase (hTERT)-HUVECs were transduced with either the Cas9-scramble sgRNA-expressing lentiviral vector (pLenti-U6-sgRNA-SFFV-Cas9-2A-Puro, # K011, ABM) or Cas9-sgDAPK3-expressing vector (# K0560905, ABM) to generate DAPK3KO immortalized HUVECs. Cells were selected with puromycin (1 μg/ml) for 7 days and subsequently plated at 0.3 cell per well into 96-well plates. All the clones used in this study were verified with Sanger sequencing for deletion and DAPK3 protein expression were assessed by Western blotting.

Immunoprecipitation and Immunoblot analysis: To prepare whole-cell lysates, the treated cells were lysed with 1×RIPA buffer (Cell Signaling) supplemented with 1 mM PMSF, 1 mM Na3VO4, and 1 mM NaF (all from Sigma). For immunoprecipitation, cells were lysed with NP-40 lysis buffer (50 mM Tris-HCl (pH 7.4), 150 mM NaCl, 2 mM EDTA, 2 mM EGTA, 1% NP-40, 10% glycerol) supplemented with the protease inhibitor and the phosphatase inhibitors above. After rotation for 30 min at 4 C, the lysates were centrifuged at 14000 rpm for 15 min. Protein concentration in the supernatants were examined with Pierce™ BCA Protein Assay Kit (Thermo Scientific).

To examine ubiquitination, HEK293T cells were transfected with expression plasmids encoding Flag-tagged STING and HA-tagged ubiquitin in the presence or absence of V5-tagged DAPK3 (wild type, D161A, or T180A mutant). At 24 h post-transfection, cells were treated with MG132 (20 μM) for 2 h and collected as described previously (Wang (2014)). Briefly, cells were lysed with 50 mM Tris-HCl (pH 7.4)/150 mM NaCl/1% SDS buffer and boiled for 15 min. The lysates were diluted 1:10 with lysis buffer (50 mM Tris-HCl (pH7.4), 150 mM NaCl, 1 mM EDTA, 1% Triton-X-100) and rotated for 30 min at 4 C. The diluted lysates were centrifuged for 15 min at 14000 rpm. The supernatants were immunoprecipitated with antibody-conjugated protein G dynabeads (Life technologies) for 2 h at 4 C. The beads were washed with 50 mM Tris-HCl (pH7.4)/1M NaCl/1 mM EDTA/1% NP-40 buffer for 5 times, resuspended with sample buffer and boiled for 5 min at 95 C. SDS-poly acrylamide gel electrophoresis (SDS-PAGE) and immunoblotting were performed.

siRNA screen of tumor suppressor genes: HUVECs were transfected in duplicate with 18, 735 individual siRNA oligonucleotide pools (from the 2007 Human siGENOME siRNA library, four siRNA oligonucleotides per pool, Dharmacon) arrayed in 384-well plates.

Immunofluorescence microscopy: Immunostaining for IRF3 nuclear translocation was performed as previously described (Zhong (2008)). Briefly, cells were fixed with 4% paraformaldehyde (PFA) (Affymetrix), permeabilized with 0.2% Triton X-100-phosphate-buffered saline (PBS), and blocked with 10% FBS/PBS. For IRF3 detection, cells were stained with primary antibodies, washed, and subsequently stained with Alexa 647 secondary antibody. DNA was counterstained with 4′,6-diamidino-2-phenylindole (DAPI). Cells were imaged on an ImageXpress Micro instrument (Molecular Device). Images were analyzed by using the enhanced translocation module of MetaXpress (Molecular Devices). Cells were scored as positive for IRF3 nuclear translocation if IRF3 fluorescence significantly overlapped that of DAPI with a correlation coefficient of between 0.6 and 0.8, adjusted according to the background fluorescence signal in untreated cells.

cGAMP measurement: HUVEC siRNA transfectants were stimulated with 1 μg/ml of poly dA:dT for 6 h. L929-mRuby-hIRF3 siRNA transfectants were stimulated with 0.5 μg/ml of poly dA:dT for 12 h. Cells were lysed with ice cold 80% methanol after wash with PBS, and intracellular cGAMP level was examined by targeting mass spectrometry.

Phosphoproteomic analysis: Stable isotope-encoded arginine and lysine. L-[13C6, 15N4]-Arginine (Silantes) and L-[13C6, 15N2]-Lysine (Silantes) were used for “heavy” labeling, and L-[12C6, 14N4]-Arginine (Sigma-Aldrich) and L-[12C6, 14N2]-Lysine ((Sigma-Aldrich) were used for “light” labeling. These amino acids were supplemented into SILAC DMEM (Thermofisher Scientific) at a final concentration of 0.073 mg/ml for Lysine and 0.042 mg/ml for Arginine. L929-mRuby-hIRF3 cells were grown in these two separate media supplemented with dialyzed FBS (Gibco) and Penicillin-Streptomycin for more than 10 passages. Then, each labeled population of cells was transduced with lentiviral shDAPK3 or shControl. After puromycin selection, the “heavy-labeled” cells were stimulated with 1 μg/ml poly dA:dT for 2 h and the “light-labeled” cells were treated with mock stimulation. Cells were lysed with 10 mM Tris-HCl (pH 7.5)/4% SDS/10 mM DTT and boiled for 10 min at 95 C and mixed together. Lysates were fractionated and digested, and phosphopeptides were enriched by TiO2/DHB or immunoprecipitation with anti-phosphotyrosine antibody [89] before LC-MS/MS analysis. All cell stimulation and handling was performed at La Jolla Institute, and the subsequent MS and data analysis (Cox (2008); Tyanova (2016)) were performed by Dr. Martin Steger, a PostDoc in the laboratory of Dr. Matthias Mann at the Max-Planck Institute, Germany.

Statistical analysis: Statistical analyses were performed with Excel (Microsoft) and Prism (GraphPad). At least three independent experiments were replicated, and error bars represent means±standard deviations. Statistical comparisons were evaluated by using ANOVA (more than 3 groups) or Student's t test (2 groups), with P values indicated in the figure legends.

### Example 1—an siRNA Screen of Tumor Suppressor Genes Identifies DAPKs as Positive Regulators of the Cytosolic DNA Sensing Pathway

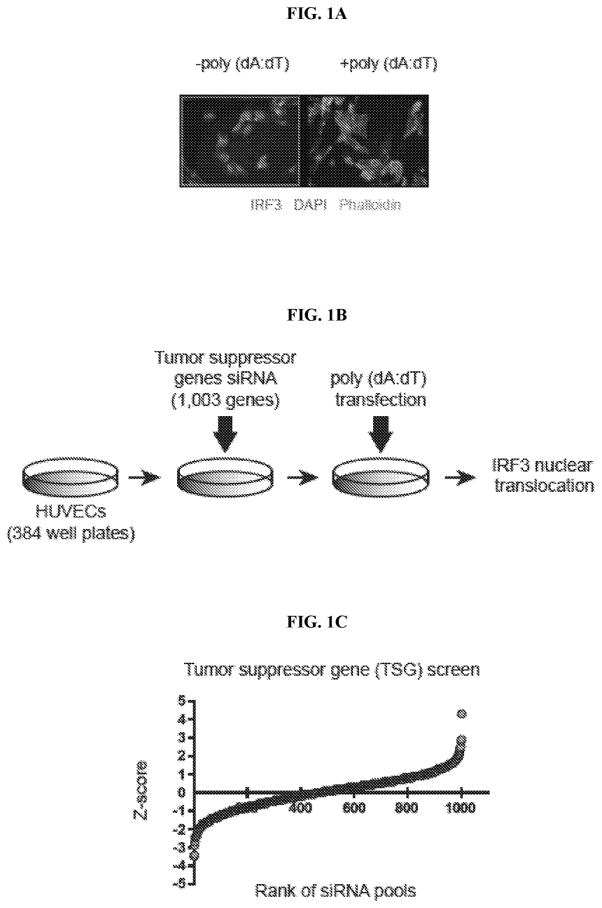

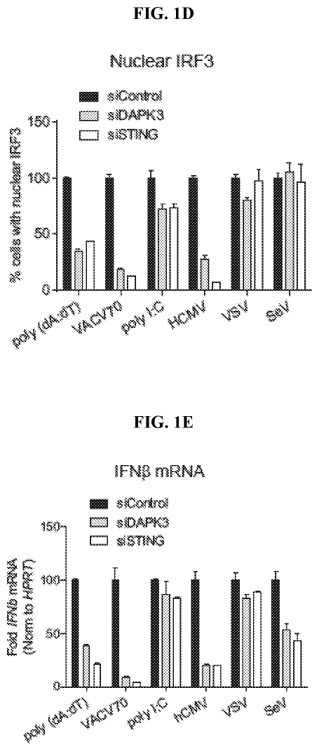

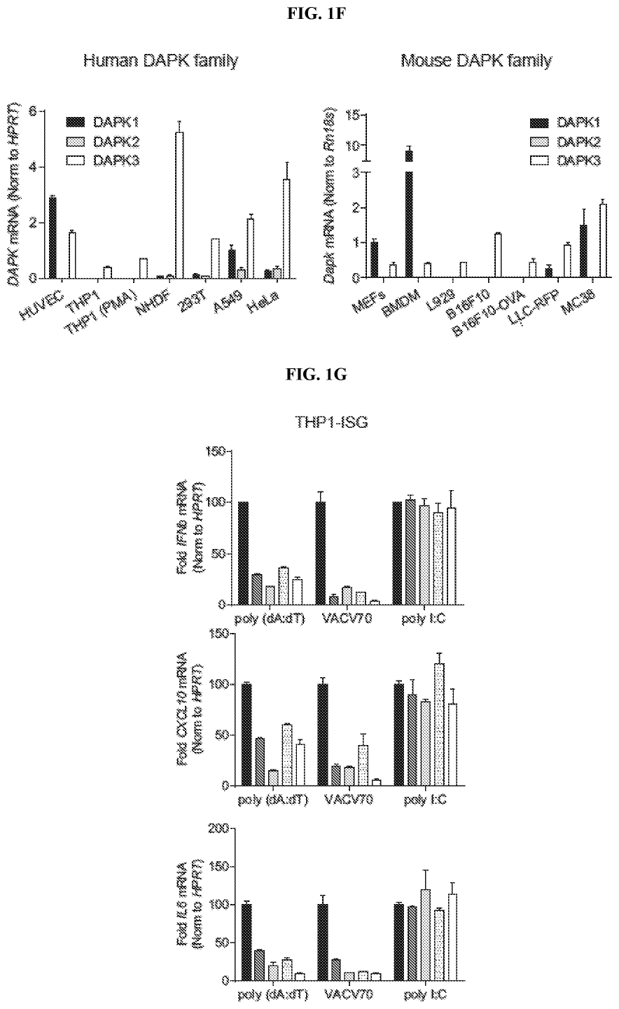

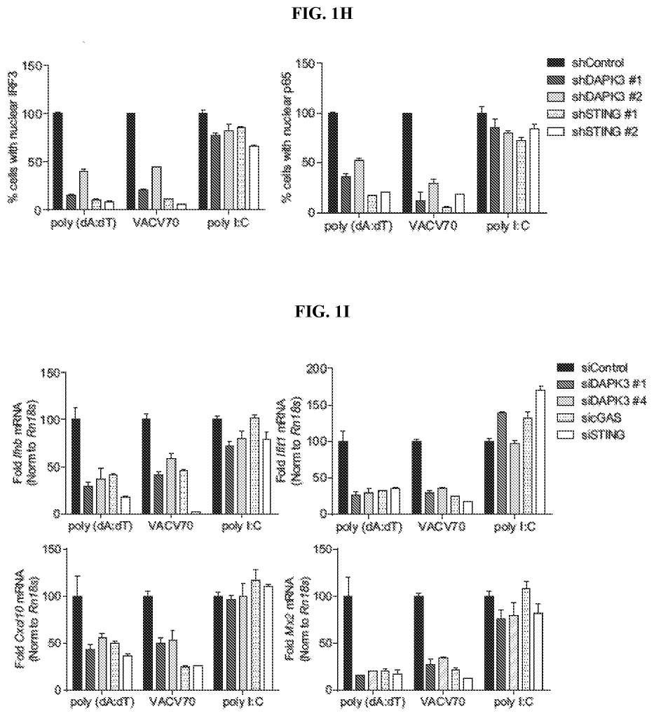

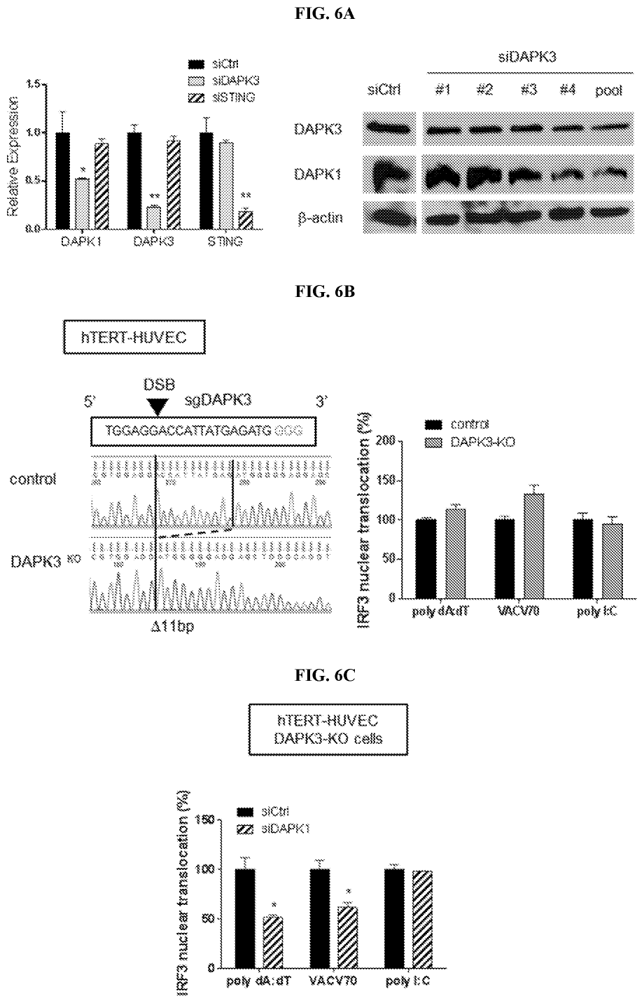

Primary human endothelial cells can mount robust innate immune responses to immuno-stimulatory DNA, which depends on cGAS, STING, and IRF3 signaling (Lio (2016)). To identify new regulators of STING-IRF3 pathway in the context of tumor immunogenecity, Applicants performed an RNAi screen of human tumor suppressor genes in human umbilical vein endothelial cells (HUVECs) using IRF3 nuclear translocation as a readout (FIGS. 1A, 1B) and identified DAPK3 as the most promising candidate (FIG. 1C). Previous studies using RNAi screen and kinome screen demonstrated that DAPK3 knockdown significantly increased hCMV replication (Terry (2012)) and VACV replication (Sivan (2013)), which support our screen results. To validate the result of the RNAi screen, primary HUVECs were transfected with pooled siDAPK3 and siSTING and examined IRF3 nuclear translocation induced by various stimuli. Depletion of DAPK3 significantly downregulated IRF3 nuclear translocation induced by immuno-stimulatory double stranded DNAs (dsDNAs), poly (dA:dT) and VACV70, vaccinia virus-derived dsDNA (FIG. 1D). On the other hand, poly LC-induced nuclear IRF3 accumulation was slightly decreased but not significantly altered in DAPK3 depleted cells compared to control cells. To examine if DAPK3 is required for virus-induced activation, DAPK3-depleted HUVECs were infected with a DNA virus, human cytomegalovirus (hCMV), and RNA viruses, vesicular stomatitis virus (VSV) and Sendai virus (SeV). hCMV-induced IRF3 translocation was markedly impaired in DAPK3-depleted cells, whereas RNA virus-induced IRF3 translocation was comparable (FIG. 1D). Similarly, DAPK3 deficiency significantly impaired DNA- and hCMV-induced transcription of IFNB1 as well as SeV-induced (FIG. 1E), which is consistent with a previous report demonstrated that STING and RIG-I interacts in HUVECs, which is by SeV infection (Ishikawa (2018)). However, when qPCR analysis was performed to check knockdown efficiency of DAPK3 and STING, it was observed that siDAPK3 used in these experiments also downregulated the mRNA level (FIG. 6A, right panel) and the protein level of DAPK1 (FIG. 6A, left panel), another DAPK family member expressed in HUVECs (FIG. 6A). Furthermore, DAPK3 knockout immortalized HUVECs generated with CRISPR/Cas9 techniques showed comparable IRF3 nuclear translocation with control cells (FIG. 6B) and DAPK1 depletion in these cells impaired activation of IRF3 induced by dsDNA (FIG. 6C), suggesting the functional redundancy of DAPK family members. qPCR analysis of DAPK family expression in human cell lines and mouse cell lines revealed that DAPK3 was ubiquitously expressed in all the cell lines tested (FIG. 1F). Some of the cell lines including HUVECs and A549, and mouse bone-marrow derived macrophages (BMDMs) and embryonic fibroblasts (MEFs) also co-expressed DAPK1. DAPK2 expression was poorly detectable in a very few cell lines, which is consistent with a previous report demonstrating that DAPK2 transcript was mainly seen in hematopoietic cells (Fang (2008)). Among the cell lines we used, human monocytic THP-1-ISG cells and mouse fibroblast L929 cells, which only express DAPK3 among DAPK family, have been widely used to examine the mechanism of how DNA sensing pathway is regulated. Then, THP-1-ISG cells were first transduced with lentiviral shDAPK3 and shSTING. DAPK3 depletion significantly impaired DNA-induced transcription of interferon-stimulated genes (ISGs) such as IFNB1, CXCL10, and IL6 in THP-1-ISG cells (FIG. 1G). To monitor IRF3 nuclear translocation in mouse cells, L929-mRuby-IRF3 cells were generated by transducing L929 cells with lentiviral mRuby-conjugated human IRF3, followed by limiting dilution-based single cell cloning (FIG. 6D). DNA-induced IRF3 and NFκB p65 nuclear translocation (FIG. 1H) and subsequent production of ISGs including Ifnb1, Ifitl, Cxcl10, Mx2, and 116 (FIG. 1I) were significantly downregulated in DAPK3-deficient L929-mRuby-hIRF3 cells. Furthermore, there was a strong correlation between dsDNA-induced nuclear IRF3 accumulation and knockdown efficiency of DAPK3 mRNA. Depletion of DAPK3 or STING slightly reduced poly I:C-induced ISG upregulation in L929-mRuby-hIRF3 cells. Previous studies showed that poly I:C and some RNA viruses facilitated RIG-I-mediated upregulation of STING (Nazmi (2012); Liu (2016)), and this result could be a secondary effect of impaired STING upregulation. Collectively, these results suggest that DAPK3 regulates the STING-IFN-I signaling pathway.

### Example 2—DAPK3 Functions at the Level of STING

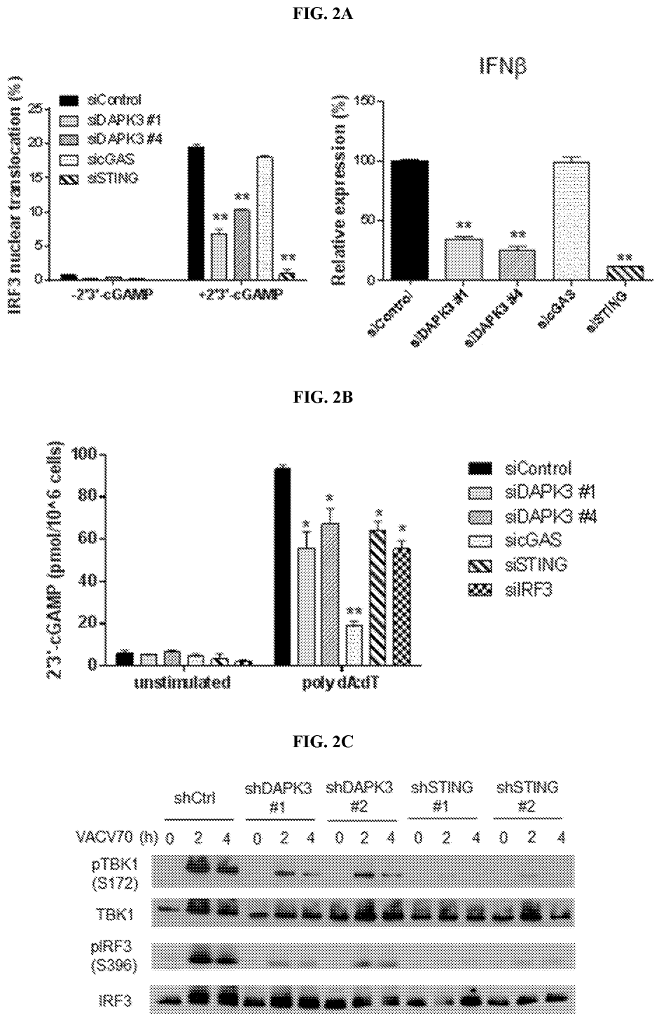

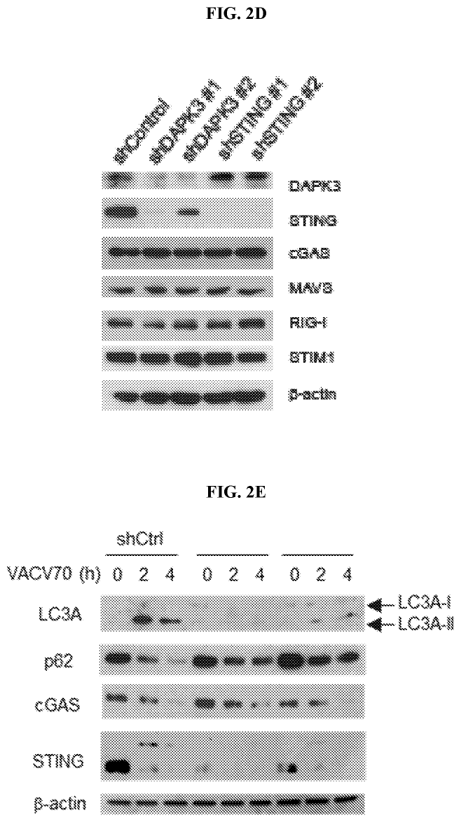

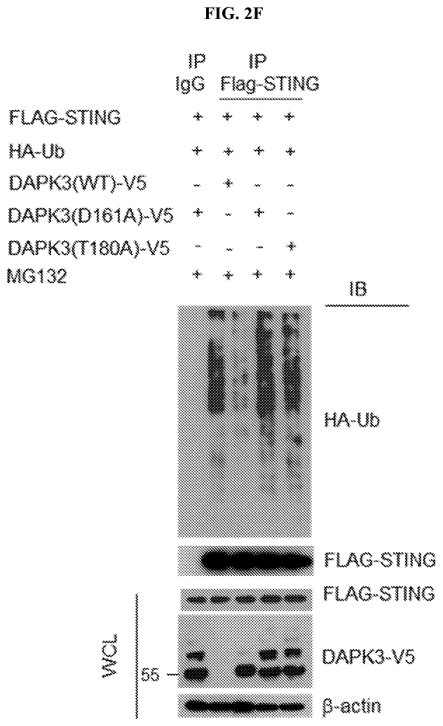

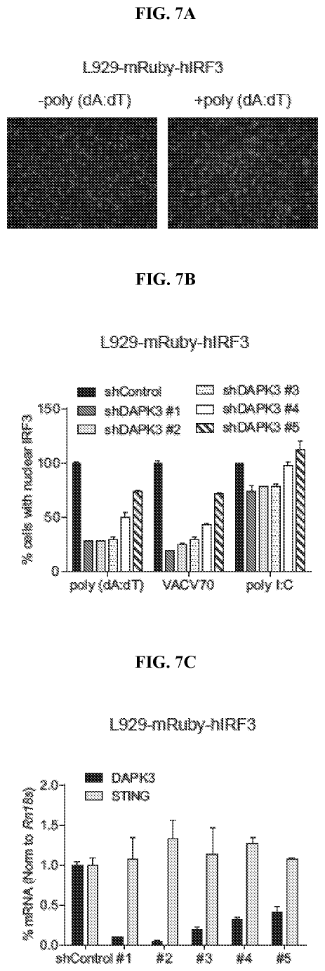

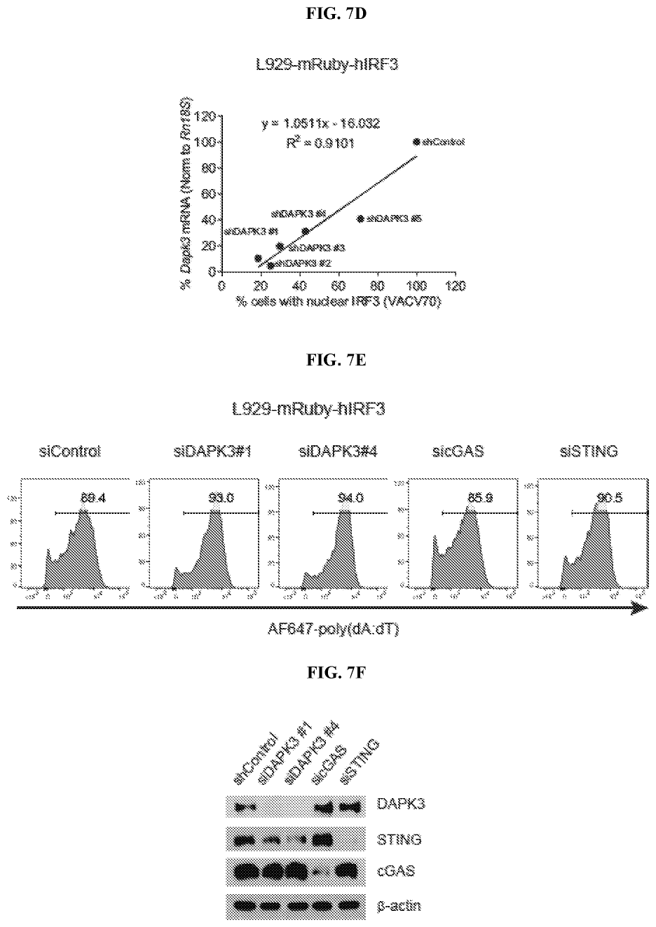

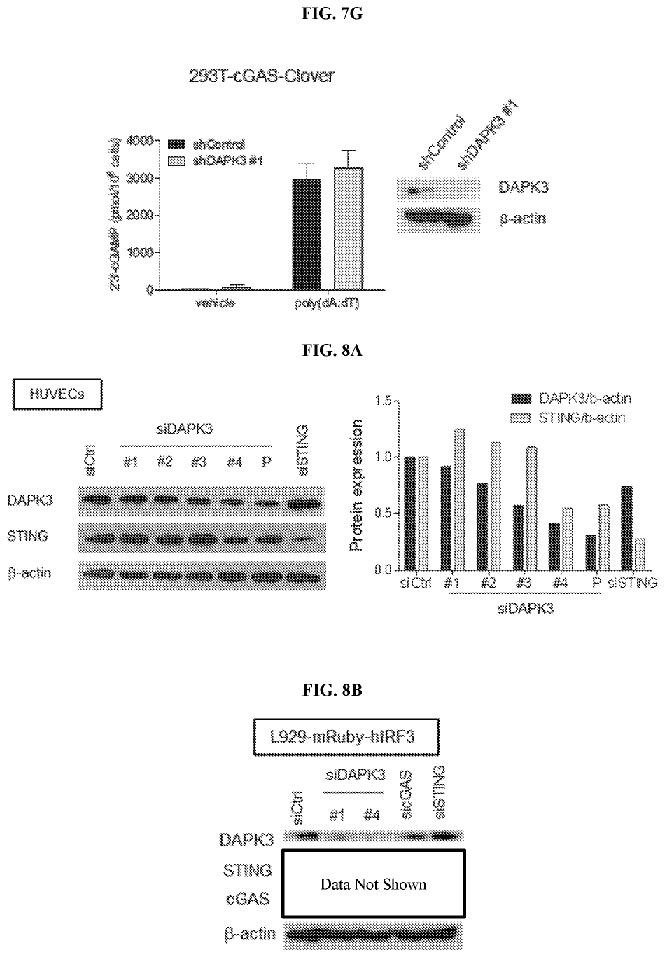

To elucidate the mechanisms by which DAPK3 is involved in DNA-induced IFN-I signaling pathway, Applicants examined if DAPK3 affected cGAMP-induced downstream activation, since DAPK3 deficiency did not significantly alter DNA uptake via endocytosis in L929-mRuby-hIRF3 cells (FIG. 7A). siRNA-transfected L929-mRuby-hIRF3 cells were stimulated with cGAMP, which bypasses the catalytic activity of cGAS, to examine IRF3 nuclear translocation and IFNB1 mRNA level induced by direct activation of STING (FIG. 2A). cGAMP-induced STING activation was markedly impaired in DAPK3-depleted cells as well as STING-depleted cells, whereas cGAS-depleted L929 cells showed comparable immune response to control cells. Next, intracellular cGAMP levels were estimated in the lysates of poly (dA:dT)-stimulated L929 cells by mass spectrometry. DAPK3 knockdown slightly downregulated cGAMP production, which was also observed in STING knockdown cells and IRF3 knockdown cells (FIG. 2B). cGAS activity has been shown to be regulated in a IFN-I dependent positive feedback loop (Ma (2015)); the decreased cGAMP production in DAPK3-deficient cells could be due to the impaired activation of downstream signaling. Similarly, cGAMP production in DAPK3-depleted HEK293T cells stably transduced with lentiviral cGAS-Clover (293T-cGAS-Clover, which lacks endogenous cGAS and STING) was comparable to control (FIGS. 7B-7D). Furthermore, phosphorylation of TBK1 and IRF3 followed by VACV70 stimulation were significantly inhibited by DAPK3 knockdown in L929-mRuby-hIRF3 cells (FIG. 2C). These results indicate that DAPK3 acts downstream of cGAS and upstream of TBK1-IRF3, possibly at the level of STING.

### Example 3—DAPK3 Maintains STING Stability in a Kinase Activity Dependent Manner

When Western blotting analysis was performed to confirm knockdown efficiency of DAPK3 and STING in shRNA-transduced cells, it was repeatedly observed that the protein expression of STING was significantly downregulated in DAPK3-depleted L929-mRuby-hIRF3 cells (FIG. 2D). DAPK3 shRNAs used did not affect the expression level of other proteins involved in regulation of nucleic sensing pathway like, cGAS, MAVS, or RIG-I. Moreover, depletion of DAPK3 in HUVECs and THP1-ISG cells also led to downregulation of STING protein (FIGS. 7E-7G). qPCR analysis revealed that STING mRNA level was comparable between shRNA and shDAPK3, suggesting that DAPK3 could regulate the expression of STING protein at a posttranscriptional level. Moreover, depletion of DAPK3 also suppressed dsDNA-induced STING-dependent autophagy, which was assessd by LC3A conversion to LC3A-II (FIG. 2E), and cGAS was comparably degraded upon stimulation in these cells. To examine the mechanism of how DAPK3 regulates the stability of STING, DAPK3-depleted L929-mRuby-hIRF3 cells were treated with various inhibitors for protein degradation pathways. Proteasome inhibitors, MG132 and Lactacystin, and autophagy inhibitors, Bafilomycin A1 and E64d+Pepstatin A, were tested and found that all the inhibitors rescued STING expression. Then, it was hypothesized that DAPK3 regulates ubiquitination of STING, upstream of proteasome-dependent degradation and selective autophagy. Previously, it was shown that knockdown of DAPK3, as well as overexpression of a dominant-negative DAPK3 mutant, diminished poly-ubiquitination of androgen receptor (Felten (2012)). To examine if DAPK3 controls ubiquitination of STING in a kinase activity-dependent manner, HEK293T cells were co-transfected with Flag-tagged STING encoding plasmid, HA-tagged ubiquitin encoding plasmid, and V5-tagged DAPK3 encoding plasmid, followed by immunoprecipitation assay with anti-Flag antibody. Wild type DAPK3 (DAPK3-WT), but not kinase dead DAPK3-D161A mutant or kinase deficient DAPK3-T180A mutant, markedly attenuated STING ubiquitination. Moreover, DAPK3 mutants showed multiple bands that have slower mobility, which were significantly less in DAPK3-WT. These results indicate that DAPK3 regulates ubiquitination of STING in a kinase activity-dependent manner and affects the function of multiple ubiquitin-related enzymes including the one that controls ubiquitination of DAPK3 itself. To investigate which types of polyubiquitin chains of STING are regulated by DAPK3, HEK293T cells were co-transfected with Flag-tagged STING and HA-tagged ubiquitin mutants (KO) in which all lysines, except for those indicated, were mutated to arginines in the presence or absence of V5-tagged DAPK3-WT, followed by immunoprecipitation assay. DAPK3 overexpression significantly downregulated K110-, K270-, K480-, or K630-linked ubiquitination of STING. Furthermore, co-transfection of Flag-tagged STING together with HA-tagged ubiquitin mutants (KR) in which a specific lysine indicated was replaced into arginine in HEK293T cells revealed that DAPK3 did not remove any of these. Taken together, these data indicate that DAPK3 controls K11, K27-, K48-, and K63-linked ubiquitination of STING. Applicants further investigated if DAPK3 kinase activity is important for dsDNA-induced innate immune responses. Kinase dead DAPK3-D161A was shown to work as a dominant negative form that disrupts DAPK3-WT activity (Brognard (2011)). Immortalized HUVECs were transduced with lentiviral luciferase, DAPK3-D161A, or DAPK3-T180A and found that overexpression of these mutants led to a significant decrease of dsDNA-IRF3 nuclear accumulation compared to luciferase overexpression. Similarly, reconstitution of DAPK3-WT in DAPK3-deficient L929-mRuby-hIRF3 cells were able to rescue nuclear IRF3 translocation induced by transfection of dsDNA, but not ds RNA. Expression of these proteins were confirmed by Western blot analysis. Collectively, these data clearly suggest that DAPK3 kinase activity is essential for regulation of dsDNA-induced innate immune response. Since DAPK3 itself doesn't have an ubiquitin ligase activity or a deubiqutinase activity, Applicants examined if DAPK3 directly phosphorylates STING using in vitro kinase assay, and found that DAPK3 did not regulate STING phosphorylation. Previous studies have shown that TBK1 phosphorylates STING at 5358 (Zhong (2008); Tanaka (2012)) and S366, which is critical for IRF3 recruitment (Liu (2015)), and possibly at 5355 (Tsuchiya (2016)). HEK293T were co-transfected cells with HA-tagged STING encoding plasmid, Flag-tagged TBK1 encoding plasmid, and V5-tagged DAPK3 plasmid, followed by immunoprecipitation with anti-HA antibody. Mass spectrometric analysis showed that 5322 and S358 of STING are phosphorylated by kinase active TBK1 but DAPK3 did not affect phosphorylation of STING even in the presence of TBK1. Taken all together, it is hypothesized that kinase active DAPK3 regulates ubiquitin-related enzymes that control STING ubiquitination directly.

### Example 4—DAPK3 Regulates Phosphorylation of Multiple Proteins Activated by Immunostimulatory dsDNA

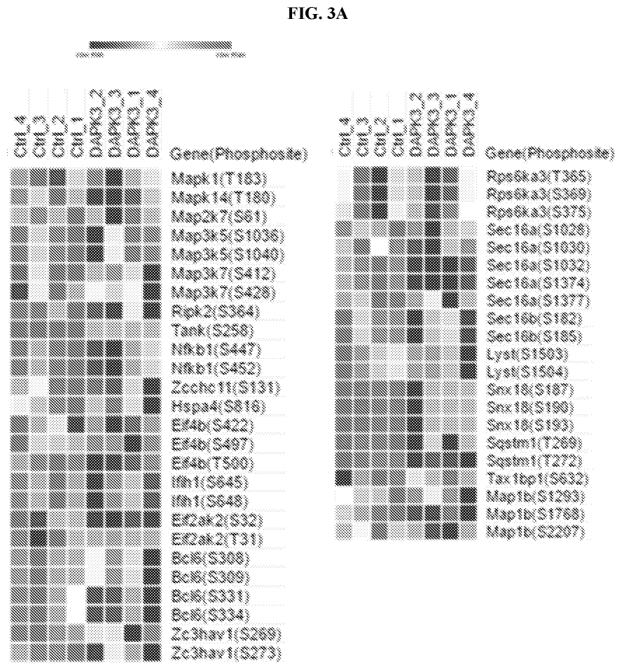

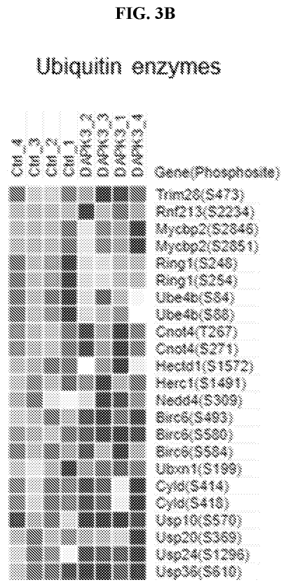

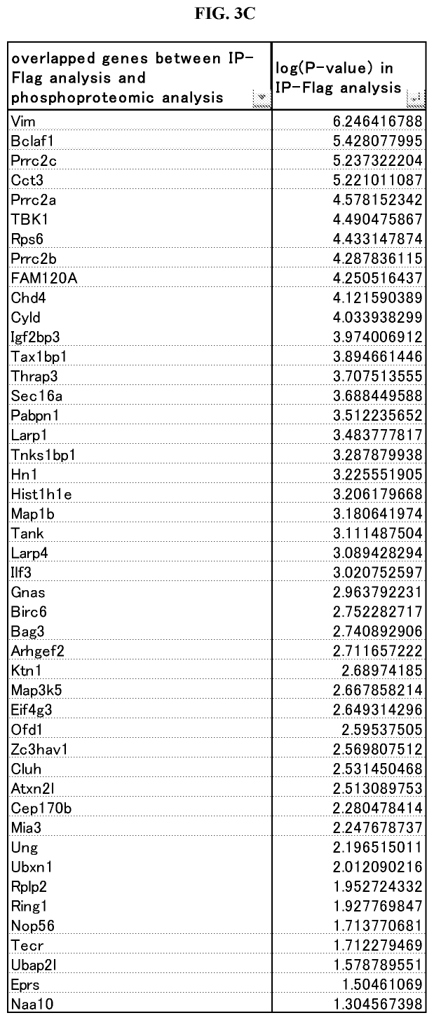

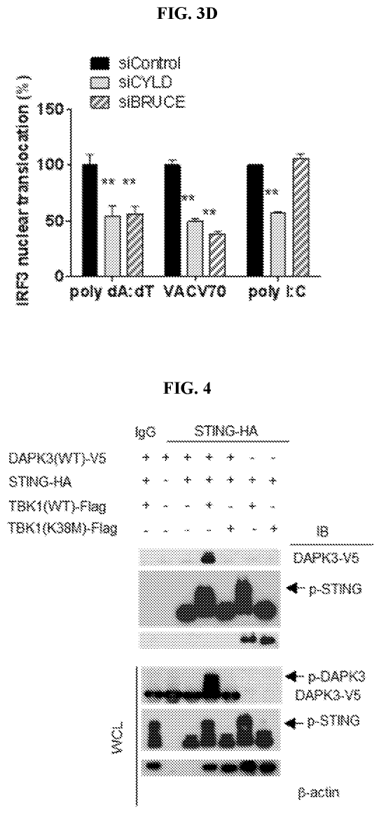

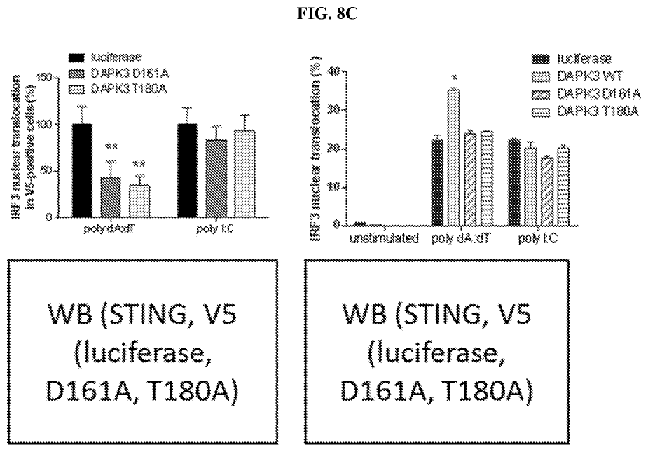

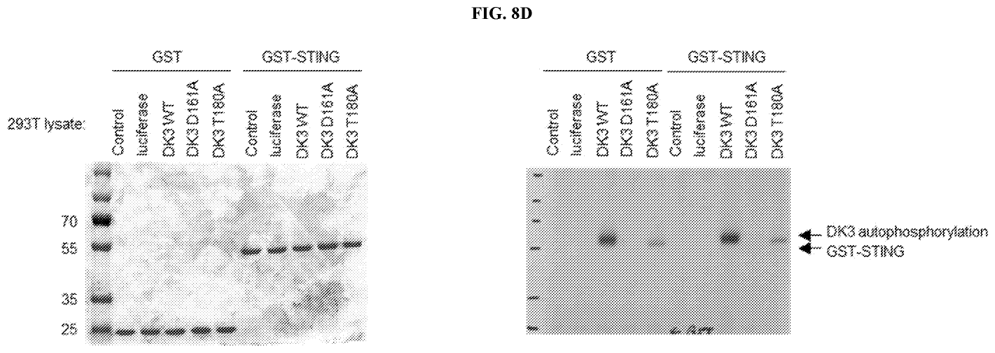

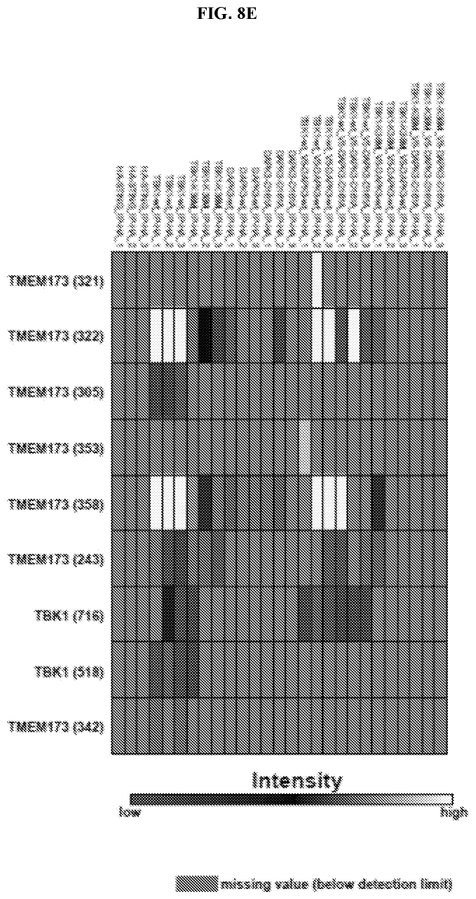

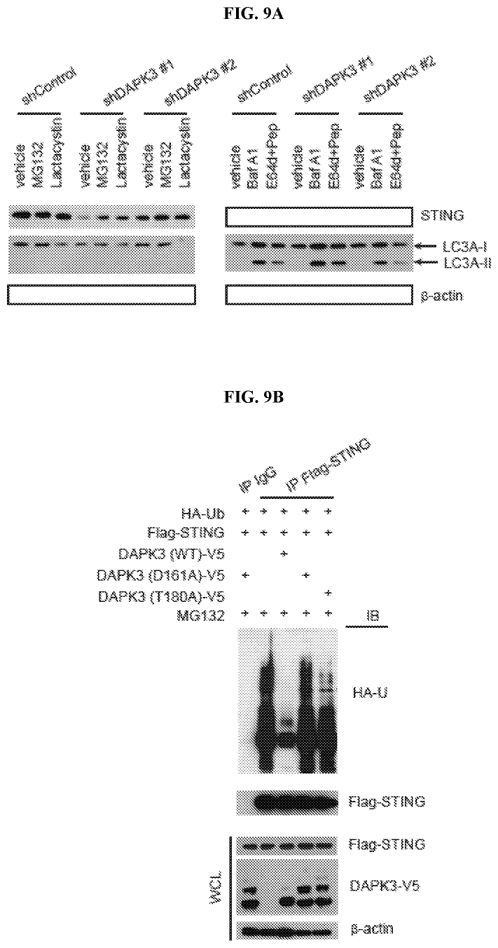

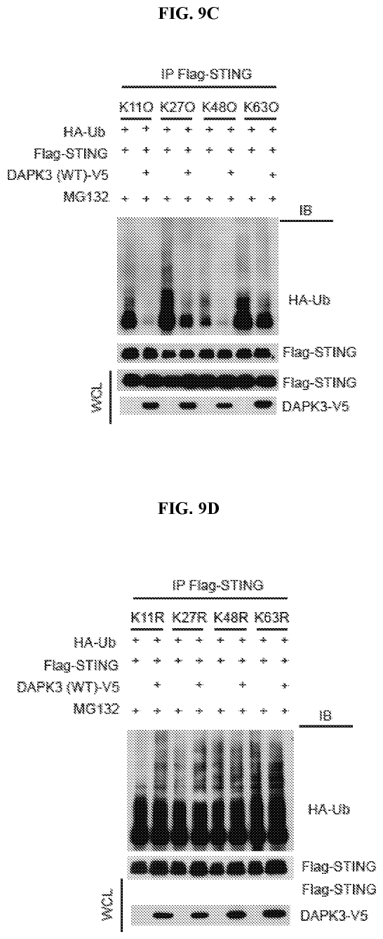

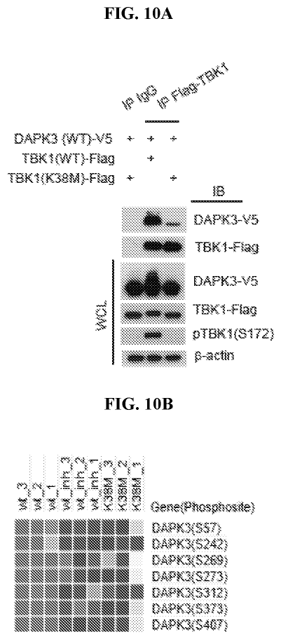

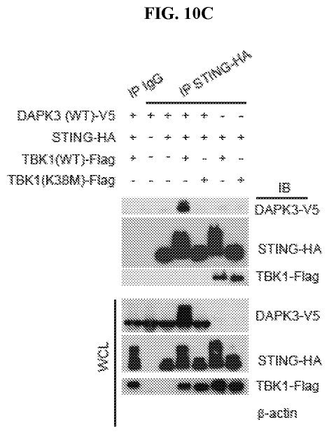

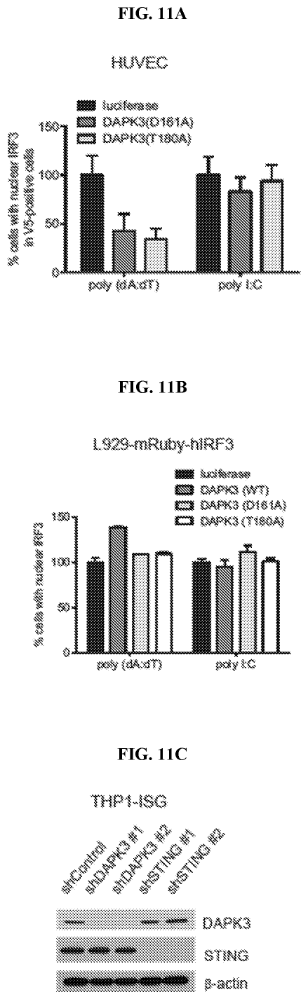

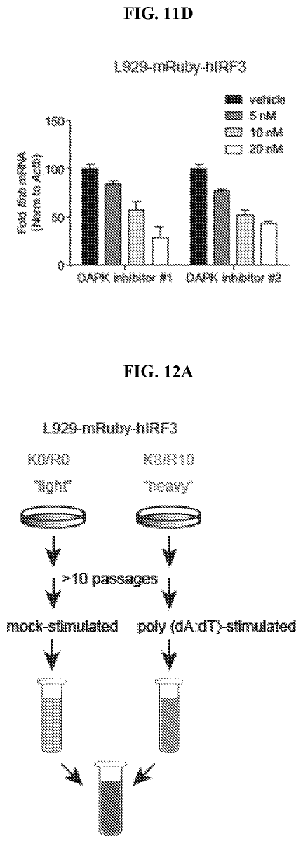

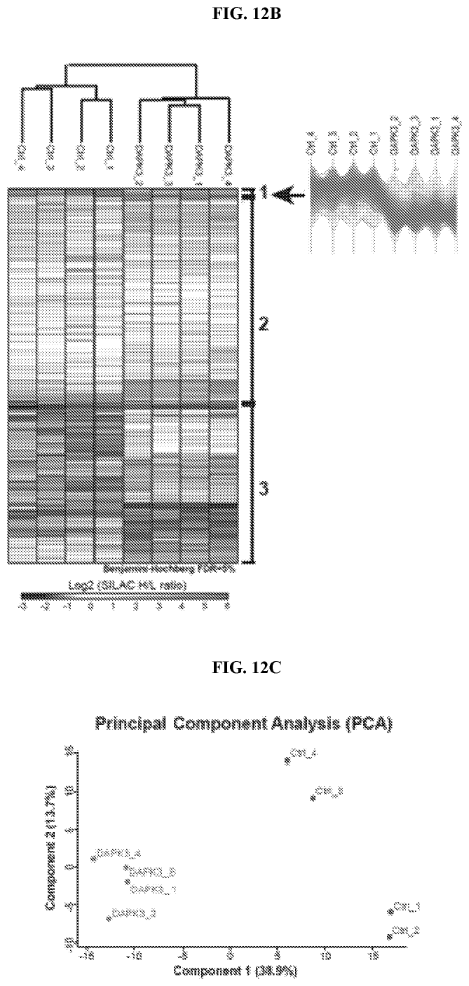

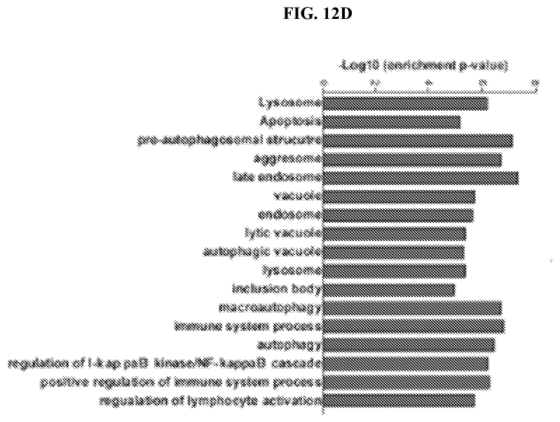

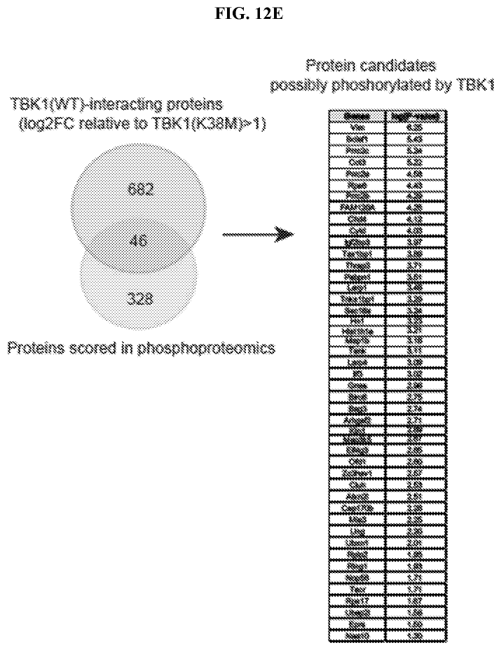

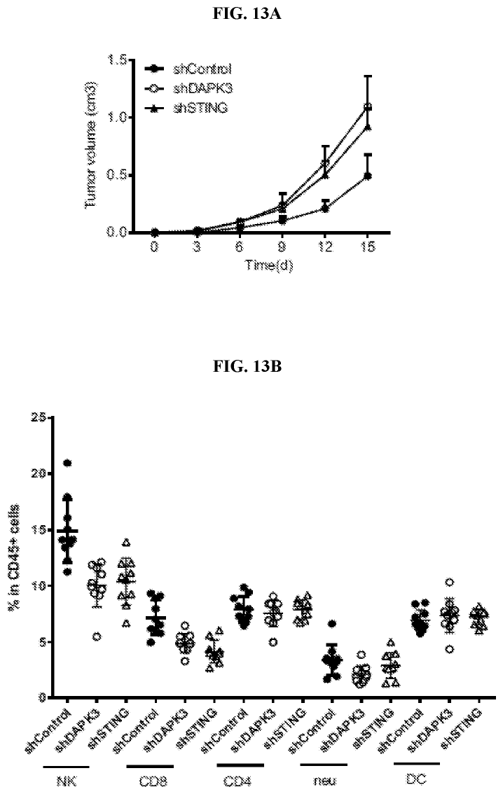

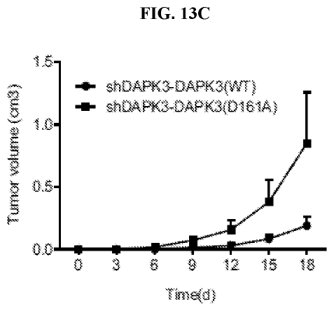

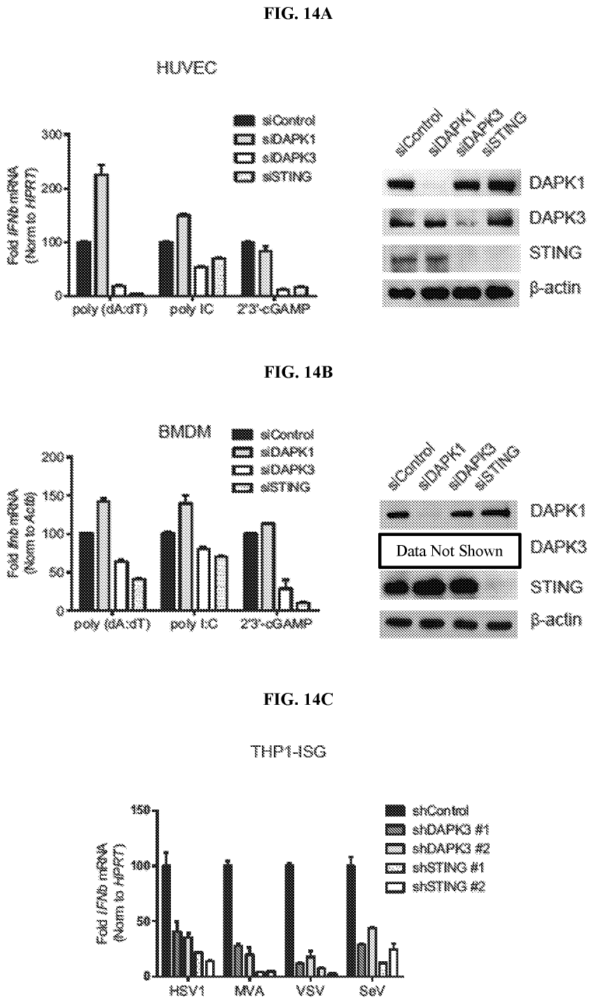

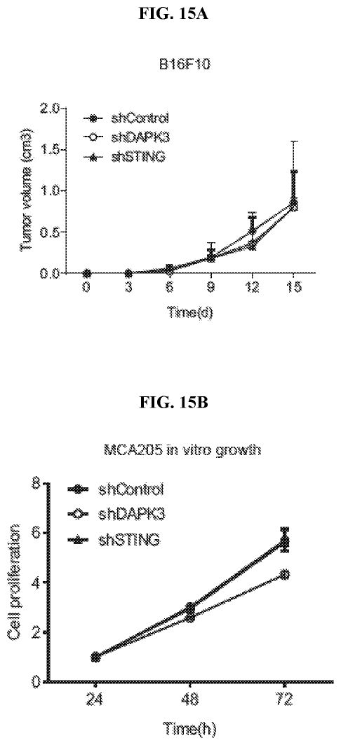

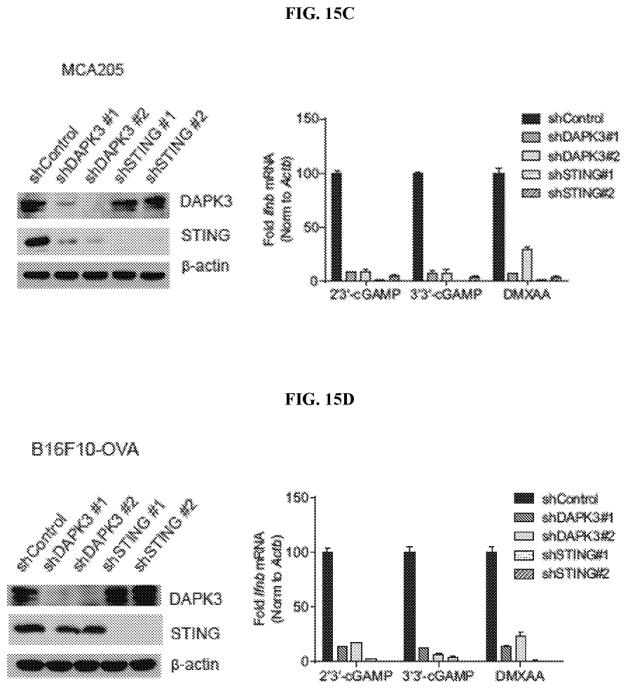

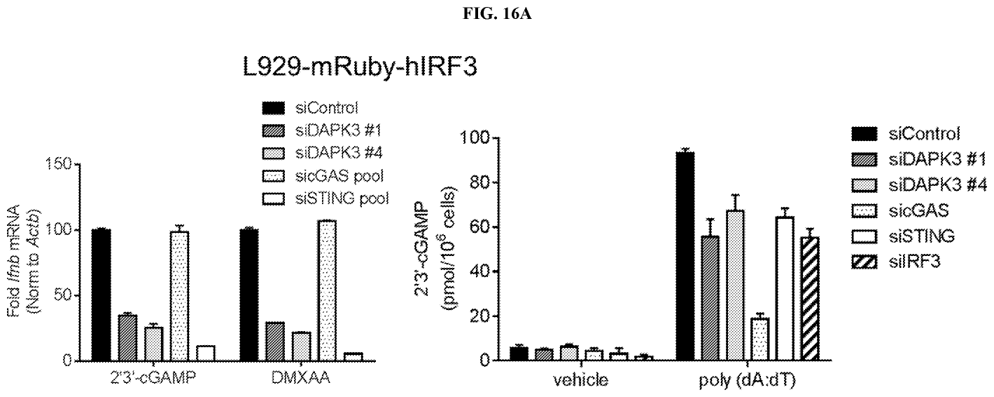

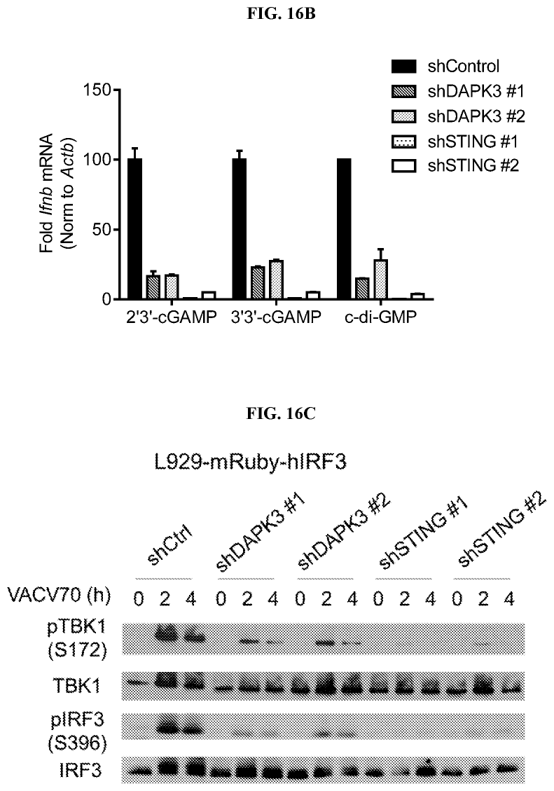

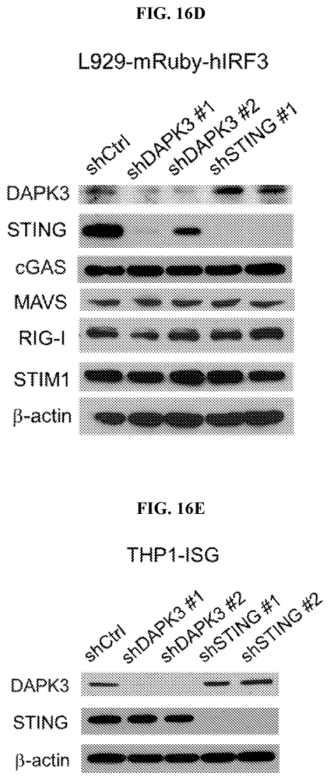

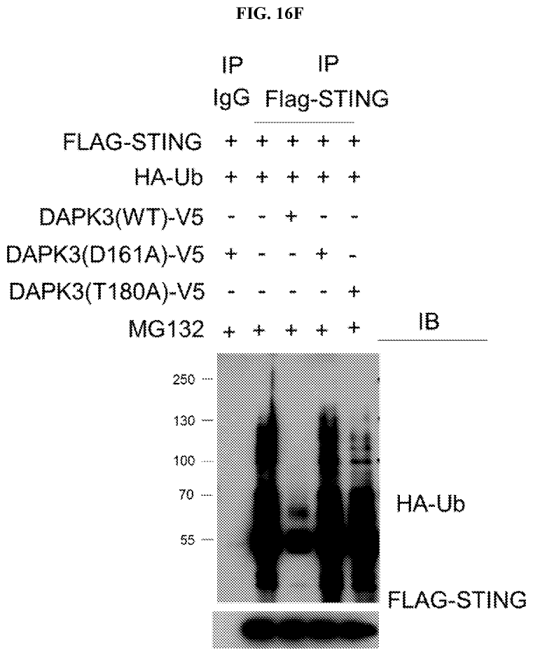

Since DAPK3 depletion caused downregulation of STING protein level in resting cells, it remains unclear what DAPK3 does in dsDNA-activated cells. To examine the phosphorylation targets of DAPK3 upon DNA stimulation, Applicants performed global phosphoproteomic analysis using stable isotope labeling of cells in culture (SILAC)-based mass spectrometry (Macek (2009)). L929-mRuby-hIRF3 cells labeled with heavy amino acids-containing media were stimulated with poly dA:dT and the other cell populations labeled with light amino acids-containing media were stimulated with mock treatment after lentiviral shRNA transduction, and the lysates were mixed together 1:1. Phosphopeptides were enriched with TiO₂ and analyzed by liquid chromatography-tandem MS (LC-MS/MS). A comparable number of phosphopeptides in DAPK3-depleted cells and control cells were quantified, and principle component analysis (PCA) revealed that there was a strong separation between these cell populations (FIG. 8A). Then Applicants focused on a gene cluster which showed higher phosphorylation upon dsDNA stimulation in control cells but less in DAPK3-depleted cells (FIG. 12B). Gene Ontology (GO) annotations were used to identify associations between proteins phosphorylated induced by dsDNA stimulation higher in control cells than in DAPK3-deficient cells and specific gene function descriptions. GO enrichment analysis revealed that genes that positively regulate the innate immune responses, NFκB and IRF signaling, endosomal-lysosomal pathway and autophagy process were highly enriched in this cluster (FIG. 3A, FIGS. 12C-12D), though phosphorylation of STING, TBK1, or IRF3 were not scored in this analysis, which could be due to the insufficient coverage. Interestingly, proteins that are activated in response to RNA sensing pathway, like melanoma-differentiation associated protein 5 (MDA5) and protein kinase R (PKR), also scored in this cluster, but most of these phosphosites are not conserved in human and do not fit DAPK3 consensus phosphorylation site, R-X-X-S (wherein x is any amino acid) (Burch (2004); Arif (2012)), and these proteins are not likely to be directly phosphorylated by DAPK3. Among these scored genes, phosphorylation of some of ubiquitin ligases and deubiquitinases (DUBs) were impaired by DAPK3 knockdown (FIG. 3B). Intriguingly, phosphorylation of CYLD, a well-known negative regulator of NFκB signaling by removing K63-linked poly-ubiquitin chains from many innate immune signaling molecules including tumor necrosis factor (TNF) receptor (TNFR)-associated factor (TRAF) family, NFκB essential modulator (NEMO, also known as IKKy) (Brummelkamp (2003); Kovalenko (2003); Trompouki (2003); Yoshida (2005); Jin (2008)), transforming growth factor-β (TGFβ) activated kinase 1 (TAK1) (Reiley (2007)), and receptor-interacting protein 1 (RIP1) (Wright (2007)), was induced upon stimulation of immunostimulatory dsDNA, which was dramatically downregulated in DAPK3-depleted cells (FIG. 3B). Previous studies have shown the important role of CYLD in RIG-I mediated antiviral signaling in vitro and in vivo (Friedman (2008); Zhang (2008); Zhang (2011)). S418 phosphorylation has been shown to be regulated by IKK family members including IKKε (Hutti (2009); Reiley (2005)) and CaMKII (Thein (2014)). An immunoprecipition assay was performed using cell lysates of HEK293T cells transfected with expression plasmids encoding V5-tagged DAPK3 and Flag-tagged TBK1 WT or K38M, and found that DAPK3 interacts with TBK1 regardless of TBK1 kinase activity (FIG. 10A). Moreover, DAPK3 bands showed slower mobility in the presence of kinase active TBK1, but not kinase dead TBK1-K38M mutant, suggesting that DAPK3 is phosphorylated by TBK1. To further confirm which amino acids of DAPK3 are targets for TBK1-mediated phosphorylation, quantitative mass spectrometry was performed using HEK293T lysates. Co-transfection of wild type TBK1 and kinase dead DAPK3 mutant D161A into HEK293T cells, followed by co-immunoprecipitation with anti-Flag antibody revealed that S57, 5269, 5273, 5288, 5312, 5373, and 5407 of DAPK3 were largely phosphorylated, which was markedly downregulated by TBK1 inhibitor BX795 or co-transfection with kinase dead TBK1-K38M mutant (FIG. 10A). Notably, S269 and S273 fits canonical target site of IκB kinase (IKKβ) (Schmid (2008)), and phosphorylation of these amino acids might be involved in regulation of DAPK3 kinase activity. Applicants also examined what kind of endogenous proteins were immunoprecipitated with TBK1 in the same lysates, and found that CYLD and BIR Repeat-containing Ubiquitin-Conjugating Enzyme (BRUCE, also known as Apollon, encoded by BIRC6) interacted with TBK1 with high affinity (FIG. 3C). Moreover, some STING regulators that were previously reported, like an E3 ligase RNF5 (Zhong (2009)), the processing body-associated protein LSM14A (Liu (2016)), and autophagy protein ATG9A (Saitoh (2009)) were also precipitated with Flag-tagged TBK1 (FIG. 8B). Since HEK293T cells do not express cGAS or STING, these results suggest that TBK1 might be involved in recruitment of these molecules to STING. Then, L929-mRuby-hIRF3 cells were transfected with siCYLD, siBRUCE, and siCtrl to examine nuclear IRF3 accumulation induced by immuno-stimulatory dsDNA and dsRNA, and the data showed that knockdown of CYLD impaired IRF3 activation induced by both stimulants, whereas BRUCE depletion downregulated only dsDNA-induced IRF3 nuclear translocation (FIG. 3D). BRUCE is a chimeric E2/E3 ligase resides in trans-Golgi network and functions as an inhibitor of apoptosis (Bartke (2004); Lotz (2004)) and a regulation of cytokinesis (Pohl (2008)). BRUCE depletion by transfection of its specific siRNA into L929-mRuby-hIRF3 cells caused significant reduction of STING protein expression at a basal level, without affecting STING mRNA level (FIG. 8C).

### Example 5—DAPK3 Interacts with STING in the Presence of Kinase Active TBK1 and Co-Localizes

Applicants next examined if DAPK3 physically interacts with STING using 293T overexpression system. Co-transfection of V5-tagged DAPK3 encoding plasmid and Flag-tagged STING encoding plasmid and immunoprecipitation assay showed that DAPK3 did not interact with STING (FIG. 4). However, in the presence of kinase active TBK1, but not kinase dead mutant TBK1-K38M, DAPK3 was associated with STING (FIG. 4). Interestingly, STING interacted with both wild type TBK1 and kinase dead K38M mutant, but phosphorylated DAPK3 disrupted STING-TBK1 interaction.

Previous studies suggest that phosphorylated STING showed s a slight shift to higher molecular weights (Gozuacik (2006)). Similar to STING, DAPK3 seemed to be phosphorylated by TBK1, as indicated by its slower mobility. The TBK1 kinase dead mutant interacted with DAPK3 slightly stronger than wild-type TBK1, indicating that interaction between DAPK3 and TBK1 was independent of TBK1 kinase activity. Consistently, the shifted bands of DAPK3 were lost when co-transfected with K38M mutant. Applicants investigated if DAPK3 interacts with TBK1 in unstimulated cells and/or DNA-activated cells. Co-immunoprecipitation of endogenous proteins in L929 cells clarifies that DAPK3 interacted with TBK1. Furthermore, confocal microscopic analysis revealed that DAPK3 and pTBK1 were well co-localized in trans-golgi network, where TBK1 interacted with dsDNA-activated STING.

### Example 6—DAPK3 Depletion in Tumor Cells Increase In Vivo Tumor Progression

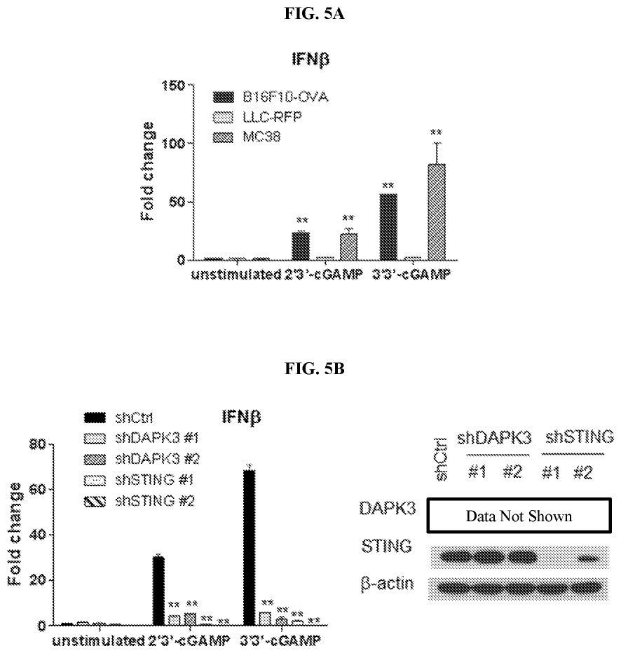

DAPK3 depletion in tumor cells impaired STING activation, leading to insufficient IFN-I and poor tumor rejection. First, several mouse tumor cell lines were stimulated with 2′3′-cGAMP and 3′3′-cGAMP, bacterial cGAMP which is widely used for in vivo injection, to see if any of the cells have potential to respond to the STING agonists (FIG. 5A). Mouse melanoma cell line B16F10-OVA and mouse colon carcinoma cell line MC38 showed significant upregulation of Ifnb1 mRNA in response to both types of cGAMP, whereas mouse lung carcinoma cell line LLC-RFP did not respond at all. To examine the role of DAPK3 in direct STING activation in tumor cells, B16F10-OVA cells were transduced with lentiviral shDAPK3 and shSTING and found that DAPK3 depletion drastically downregulated cGAMP-induced transcription of Ifnb1 as well as STING depletion (FIG. 5B, left panel). However, DAPK3 deficiency did not affect STING protein expression (FIG. 5B, right panel), suggesting that the regulation of STING protein level by DAPK3 might be cell type-specific.

### Example 7—Mouse Models

DAPK3 modulation is examined in inflammatory models, such as TREX1-deficient mice (a mouse model for spontaneous IFNb-driven systemic inflammation which is found in such diseases as Aicardi-Goutieres Syndrome (a clinical sub-type of lupus)). Expression of IFNb and other IRF3-driven cytokines is observed, as well as histology of affected tissues (e.g., heart, GI tract, kidney, liver, lungs, brain, and skin). Antagonists are screened for their efficacy in altering these endpoints and reducing inflammation. Appropriate murine models for cancer and viral infections are selected and screened for agonists in a parallel manner.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Kanneganti, T. D., Central roles of NLRs and inflammasomes in viral infection. Nat Rev Immunol, 2010. 10(10): p. 688-98.
2. Kawai, T. and S. Akira, The role of pattern-recognition receptors in innate immunity: update on Toll-like receptors. Nat Immunol, 2010. 11(5): p. 373-84.
3. Bowie, A. G. and L. Unterholzner, Viral evasion and subversion of pattern-recognition receptor signalling. Nat Rev Immunol, 2008. 8(12): p. 911-22.
4. Ishikawa, H. and G. N. Barber, STING is an endoplasmic reticulum adaptor that facilitates innate immune signalling. Nature, 2008. 455(7213): p. 674-8.
5. Jin, L., et al., MPYS, a novel membrane tetraspanner, is associated with major histocompatibility complex class II and mediates transduction of apoptotic signals. Mol Cell Biol, 2008. 28(16): p. 5014-26.
6. Zhong, B., et al., The adaptor protein MITA links virus-sensing receptors to IRF3 transcription factor activation. Immunity, 2008. 29(4): p. 538-50.
7. Ishikawa, H., Z. Ma, and G. N. Barber, STING regulates intracellular DNA-mediated, type I interferon-dependent innate immunity. Nature, 2009. 461(7265): p. 788-92.
8. Sun, W., et al., EMS, an endoplasmic reticulum IFN stimulator, activates innate immune signaling through dimerization. Proc Natl Acad Sci USA, 2009. 106(21): p. 8653-8.
9. Holm, C. K., et al., Influenza A virus targets a cGAS-independent STING pathway that controls enveloped RNA viruses. Nat Commun, 2016. 7: p. 10680.
10. Sun, L., et al., Cyclic GMP-AMP synthase is a cytosolic DNA sensor that activates the type I interferon pathway. Science, 2012. 339(6121): p. 786-91.
11. Wu, J., et al., Cyclic GMP-AMP is an endogenous second messenger in innate immune signaling by cytosolic DNA. Science, 2012. 339(6121): p. 826-30.
12. Ablasser, A., et al., cGAS produces a 2′-5′-linked cyclic dinucleotide second messenger that activates STING. Nature, 2013. 498(7454): p. 380-4.
13. Diner, E. J., et al., The innate immune DNA sensor cGAS produces a noncanonical cyclic dinucleotide that activates human STING. Cell Rep, 2013. 3(5): p. 1355-61.
14. Sharma, S., et al., Triggering the interferon antiviral response through an IKK-related pathway. Science, 2003. 300(5622): p. 1148-51.
15. Fitzgerald, K. A., et al., IKKepsilon and TBK1 are essential components of the IRF3 signaling pathway. Nat Immunol, 2003. 4(5): p. 491-6.
16. Woo, S. R., L. Corrales, and T. F. Gajewski, The STING pathway and the T cell-inflamed tumor microenvironment. Trends Immunol, 2015. 36(4): p. 250-6.
17. Zitvogel, L., et al., Type I interferons in anticancer immunity. Nat Rev Immunol, 2015. 15(7): p. 405-14.
18. Diamond, M. S., et al., Type I interferon is selectively required by dendritic cells for immune rejection of tumors. J Exp Med, 2011. 208(10): p. 1989-2003.
19. Fuertes, M. B., et al., Host type I IFN signals are required for antitumor CD8+ T cell responses through CD8{alpha}+dendritic cells. J Exp Med, 2011. 208(10): p. 2005-16.
20. Dunn, G. P., et al., A critical function for type I interferons in cancer immunoediting. Nat Immunol, 2005. 6(7): p. 722-9.
21. Swann, J. B., et al., Type I IFN contributes to NK cell homeostasis, activation, and antitumor function. J Immunol, 2007. 178(12): p. 7540-9.
22. Burnette, B. C., et al., The efficacy of radiotherapy relies upon induction of type i interferon-dependent innate and adaptive immunity. Cancer Res, 2011. 71(7): p. 2488-96.
23. Pardoll, D. M., The blockade of immune checkpoints in cancer immunotherapy. Nat Rev Cancer, 2012. 12(4): p. 252-64.
24. Corrales, L., et al., Direct Activation of STING in the Tumor Microenvironment Leads to Potent and Systemic Tumor Regression and Immunity. Cell Rep, 2015. 11(7): p. 1018-30.
25. Fu, J., et al., STING agonist formulated cancer vaccines can cure established tumors resistant to PD-1 blockade. Sci Transl Med, 2015. 7(283): p. 283ra52.
26. Deng, L., et al., STING-Dependent Cytosolic DNA Sensing Promotes Radiation-Induced Type I Interferon-Dependent Antitumor Immunity in Immunogenic Tumors. Immunity, 2014. 41(5): p. 843-52.
27. Woo, S. R., et al., STING-dependent cytosolic DNA sensing mediates innate immune recognition of immunogenic tumors. Immunity, 2014. 41(5): p. 830-42.
28. Xu, M. M., et al., Dendritic Cells but Not Macrophages Sense Tumor Mitochondrial DNA for Cross-priming through Signal Regulatory Protein alpha Signaling. Immunity, 2017. 47(2): p. 363-373 e5.
29. Demaria, O., et al., STING activation of tumor endothelial cells initiates spontaneous and therapeutic antitumor immunity. Proc Natl Acad Sci USA, 2015. 112(50): p. 15408-13.
30. Bidwell, B. N., et al., Silencing of Irf7 pathways in breast cancer cells promotes bone metastasis through immune escape. Nat Med, 2012. 18(8): p. 1224-31.
31. Sistigu, A., et al., Cancer cell-autonomous contribution of type I interferon signaling to the efficacy of chemotherapy. Nat Med, 2014. 20(11): p. 1301-9.
32. Andzinski, L., et al., Growing tumors induce a local STING dependent Type I IFN response in dendritic cells. Int J Cancer, 2016. 139(6): p. 1350-7.
33. Xia, T., et al., Deregulation of STING Signaling in Colorectal Carcinoma Constrains DNA Damage Responses and Correlates With Tumorigenesis. Cell Rep, 2016. 14(2): p. 282-97.
34. Song, S., et al., Decreased expression of STING predicts poor prognosis in patients with gastric cancer. Sci Rep, 2017. 7: p. 39858.
35. Inbal, B., et al., DAP kinase links the control of apoptosis to metastasis. Nature, 1997. 390(6656): p. 180-4.
36. Geering, B., Death-associated protein kinase 2: Regulator of apoptosis, autophagy and inflammation. Int J Biochem Cell Biol, 2015. 65: p. 151-4.
37. Kawai, T., et al., ZIP kinase, a novel serine/threonine kinase which mediates apoptosis. Mol Cell Biol, 1998. 18(3): p. 1642-51.
38. Bialik, S. and A. Kimchi, The death-associated protein kinases: structure, function, and beyond. Annu Rev Biochem, 2006. 75: p. 189-210.
39. Gozuacik, D. and A. Kimchi, DAPk protein family and cancer. Autophagy, 2006. 2(2): p. 74-9.
40. Raveh, T. and A. Kimchi, DAP kinase-a proapoptotic gene that functions as a tumor suppressor. Exp Cell Res, 2001. 264(1): p. 185-92.
41. Katzenellenbogen, R. A., S. B. Baylin, and J. G. Herman, Hypermethylation of the DAP-kinase CpG island is a common alteration in B-cell malignancies. Blood, 1999. 93(12): p. 4347-53.
42. Tang, X., et al., Hypermethylation of the death-associated protein (DAP) kinase promoter and aggressiveness in stage I non-small-cell lung cancer. J Natl Cancer Inst, 2000. 92(18): p. 1511-6.
43. Mittag, F., et al., DAPK promotor methylation is an early event in colorectal carcinogenesis. Cancer Lett, 2006. 240(1): p. 69-75.
44. Brognard, J., et al., Cancer-associated loss-of-function mutations implicate DAPK3 as a tumor-suppressing kinase. Cancer Res, 2011. 71(8): p. 3152-61.
45. Lai, M. Z. and R. H. Chen, Regulation of inflammation by DAPK. Apoptosis, 2013. 19(2): p. 357-63.
46. Usui, T., M. Okada, and H. Yamawaki, Zipper interacting protein kinase (ZIPK): function and signaling. Apoptosis, 2013. 19(2): p. 387-91.
47. Zhang, J., et al., Death-associated protein kinase 1 is an IRF3/7-interacting protein that is involved in the cellular antiviral immune response. Cell Mol Immunol, 2014. 11(3): p. 245-52.
48. Willemsen, J., et al., Phosphorylation-Dependent Feedback Inhibition of RIG-I by DAPK1 Identified by Kinome-wide siRNA Screening. Mol Cell, 2017. 65(3): p. 403-415 e8.
49. Lio, C. W., et al., cGAS-STING Signaling Regulates Initial Innate Control of Cytomegalovirus Infection. J Virol, 2016. 90(17): p. 7789-97.
50. Terry, L. J., et al., Human kinome profiling identifies a requirement for AMP-activated protein kinase during human cytomegalovirus infection. Proc Natl Acad Sci USA, 2012. 109(8): p. 3071-6.
51. Sivan, G., et al., Human genome-wide RNAi screen reveals a role for nuclear pore proteins in poxvirus morphogenesis. Proc Natl Acad Sci USA, 2013. 110(9): p. 3519-24.
52. Fang, J., et al., Attenuation of EPO-dependent erythroblast formation by death-associated protein kinase-2. Blood, 2008. 112(3): p. 886-90.
53. Nazmi, A., et al., STING mediates neuronal innate immune response following Japanese encephalitis virus infection. Sci Rep, 2012. 2: p. 347.
54. Liu, Y., et al., RIG-I-Mediated STING Upregulation Restricts Herpes Simplex Virus 1 Infection. J Virol, 2016. 90(20): p. 9406-19.
55. Ma, F., et al., Positive feedback regulation of type I IFN production by the IFN-inducible DNA sensor cGAS. J Immunol, 2015. 194(4): p. 1545-54.
56. Felten, A., et al., Zipper-interacting protein kinase is involved in regulation of ubiquitination of the androgen receptor, thereby contributing to dynamic transcription complex assembly. Oncogene, 2012. 32(41): p. 4981-8.
57. Tanaka, Y. and Z. J. Chen, STING specifies IRF3 phosphorylation by TBK1 in the cytosolic DNA signaling pathway. Sci Signal, 2012. 5(214): p. ra20.
58. Liu, S., et al., Phosphorylation of innate immune adaptor proteins MAVS, STING, and TRIF induces IRF3 activation. Science, 2015. 347(6227): p. aaa2630.
59. Tsuchiya, Y., et al., Ligand-induced Ordering of the C-terminal Tail Primes STING for Phosphorylation by TBK1. EBioMedicine, 2016. 9: p. 87-96.
60. Macek, B., M. Mann, and J. V. Olsen, Global and site-specific quantitative phosphoproteomics: principles and applications. Annu Rev Pharmacol Toxicol, 2009. 49: p. 199-221.
61. Burch, L. R., et al., Phage-peptide display identifies the interferon-responsive, death-activated protein kinase family as a novel modifier of MDM2 and p21WAF1. J Mol Biol, 2004. 337(1): p. 115-28.
62. Arif, A., et al., Heterotrimeric GAIT complex drives transcript-selective translation inhibition in murine macrophages. Mol Cell Biol, 2012. 32(24): p. 5046-55.
63. Brummelkamp, T. R., et al., Loss of the cylindromatosis tumour suppressor inhibits apoptosis by activating NF-kappaB. Nature, 2003. 424(6950): p. 797-801.
64. Kovalenko, A., et al., The tumour suppressor CYLD negatively regulates NF-kappaB signalling by deubiquitination. Nature, 2003. 424(6950): p. 801-5.
65. Trompouki, E., et al., CYLD is a deubiquitinating enzyme that negatively regulates NF-kappaB activation by TNFR family members. Nature, 2003. 424(6950): p. 793-6.
66. Yoshida, H., et al., The tumor suppressor cylindromatosis (CYLD) acts as a negative regulator for toll-like receptor 2 signaling via negative cross-talk with TRAF6 AND TRAF7. J Biol Chem, 2005. 280(49): p. 41111-21.
67. Jin, W., et al., Deubiquitinating enzyme CYLD negatively regulates RANK signaling and osteoclastogenesis in mice. J Clin Invest, 2008. 118(5): p. 1858-66.
68. Reiley, W. W., et al., Deubiquitinating enzyme CYLD negatively regulates the ubiquitin-dependent kinase Takl and prevents abnormal T cell responses. J Exp Med, 2007. 204(6): p. 1475-85.
69. Wright, A., et al., Regulation of early wave of germ cell apoptosis and spermatogenesis by deubiquitinating enzyme CYLD. Dev Cell, 2007. 13(5): p. 705-16.
70. Friedman, C. S., et al., The tumour suppressor CYLD is a negative regulator of RIG-I-mediated antiviral response. EMBO Rep, 2008. 9(9): p. 930-6.
71. Zhang, M., et al., Regulation of IkappaB kinase-related kinases and antiviral responses by tumor suppressor CYLD. J Biol Chem, 2008. 283(27): p. 18621-6.
72. Zhang, M., et al., Regulation of antiviral innate immunity by deubiquitinase CYLD. Cell Mol Immunol, 2011. 8(6): p. 502-4.
73. Hutti, J. E., et al., Phosphorylation of the tumor suppressor CYLD by the breast cancer oncogene IKKepsilon promotes cell transformation. Mol Cell, 2009. 34(4): p. 461-72.
74. Reiley, W., et al., Regulation of the deubiquitinating enzyme CYLD by IkappaB kinase gamma-dependent phosphorylation. Mol Cell Biol, 2005. 25(10): p. 3886-95.
75. Thein, S., et al., CaMKII mediates recruitment and activation of the deubiquitinase CYLD at the postsynaptic density. PLoS One, 2014. 9(3): p. e91312.
76. Schmid, J. A. and A. Birbach, IkappaB kinase beta (IKKbeta/IKK2/IKBKB)—a key molecule in signaling to the transcription factor NF-kappaB. Cytokine Growth Factor Rev, 2008. 19(2): p. 157-65.
77. Zhong, B., et al., The ubiquitin ligase RNF5 regulates antiviral responses by mediating degradation of the adaptor protein MITA. Immunity, 2009. 30(3): p. 397-407.
78. Liu, T. T., et al., LSm14A Plays a Critical Role in Antiviral Immune Responses by Regulating MITA Level in a Cell-Specific Manner. J Immunol, 2016. 196(12): p. 5101-11.
79. Saitoh, T., et al., Atg9a controls dsDNA-driven dynamic translocation of STING and the innate immune response. Proc Natl Acad Sci USA, 2009. 106(49): p. 20842-6.
80. Bartke, T., et al., Dual role of BRUCE as an antiapoptotic IAP and a chimeric E2/E3 ubiquitin ligase. Mol Cell, 2004. 14(6): p. 801-11.
81. Lotz, K., G. Pyrowolakis, and S. Jentsch, BRUCE, a giant E2/E3 ubiquitin ligase and inhibitor of apoptosis protein of the trans-Golgi network, is required for normal placenta development and mouse survival. Mol Cell Biol, 2004. 24(21): p. 9339-50.
82. Pohl, C. and S. Jentsch, Final stages of cytokinesis and midbody ring formation are controlled by BRUCE. Cell, 2008. 132(5): p. 832-45.
83. Hornung, V., et al., AIM2 recognizes cytosolic dsDNA and forms a caspase-1-activating inflammasome with ASC. Nature, 2009. 458(7237): p. 514-8.
84. Imanishi, T., et al., Nucleic acid sensing by T cells initiates Th2 cell differentiation. Nat Commun, 2014. 5: p. 3566.
85. Johannessen, C. M., et al., COT drives resistance to RAF inhibition through MAP kinase pathway reactivation. Nature, 2010. 468(7326): p. 968-72.
86. Campeau, E., et al., A versatile viral system for expression and depletion of proteins in mammalian cells. PLoS One, 2009. 4(8): p. e6529.
87. Stegmeier, F., et al., The tumor suppressor CYLD regulates entry into mitosis. Proc Natl Acad Sci USA, 2007. 104(21): p. 8869-74.
88. Wang, Q., et al., The E3 ubiquitin ligase AMFR and INSIG1 bridge the activation of TBK1 kinase by modifying the adaptor STING. Immunity, 2014. 41(6): p. 919-33.
89. Humphrey, S. J., S. B. Azimifar, and M. Mann, High-throughput phosphoproteomics reveals in vivo insulin signaling dynamics. Nat Biotechnol, 2015. 33(9): p. 990-5.
90. Cox, J. and M. Mann, MaxQuant enables high peptide identification rates, individualized p.p.b.-range mass accuracies and proteome-wide protein quantification. Nat Biotechnol, 2008. 26(12): p. 1367-72.
91. Tyanova, S., et al., The Perseus computational platform for comprehensive analysis of (prote)omics data. Nat Methods, 2016. 13(9): p. 731-40.

1. A method of enhancing an innate immune response in a subject comprising administering an effective amount of an agonist of the interaction between a death associated protein kinase (DAPK) and stimulator of interferon genes protein (STING) pathway.

2. The method of claim 1, wherein the agonist is a protein, peptide, small molecule, antibody, bispecific antibody, antibody derivative, ligand mimetic, nucleic acid, or pharmaceutical composition.

3. The method of claim 1, wherein the agonist upregulates DAPK expression.

4. The method of claim 3, wherein the agonist is a vector comprising a polynucleotide encoding DAPK.

5. The method of any one of claims 1 to 4, wherein the agonist is an agonist of the interaction between DAPK and TBK1 and/or STING.

6. The method of any one of claims 1 to 5, wherein the subject is a mammal.

7. The method of claim 6, wherein the mammal is a human.

8. The method of any one of claims 1 to 7, wherein the DAPK is DAPK1, DAPK2, or DAPK3.

9. The method of any one of claims 1 to 8, wherein the subject has a viral infection.

10. The method of any one of claims 1 to 8, wherein the subject has cancer, optionally wherein the agonist is specifically administered to a tumor cell.

11. The method of claim 10, wherein the cancer is melanoma or colon carcinoma.

12. The method of any one of claims 1 to 11, wherein the agonist is specifically administered to one or more of endothelial cells, monocytes, or fibroblasts.

13. A method of increasing type I interferon (IFN-I) expression in a population of cells comprising administering an effective amount of an agonist of the interaction between DAPK and STING pathway to the population of cells.

14. The method of claim 13, wherein the agonist is a protein, peptide, small molecule, antibody, bispecific antibody, antibody derivative, ligand mimetic, nucleic acid, or pharmaceutical composition.

15. The method of claim 13, wherein the agonist upregulates DAPK expression.

16. The method of claim 15, wherein the agonist is a vector comprising a polynucleotide encoding DAPK.

17. The method of any one of claims 13 to 16, wherein the agonist is an agonist of the interaction between DAPK and TBK1 and/or STING.

18. The method of any one of claims 13 to 17, wherein the DAPK is DAPK1, DAPK2, or DAPK3.

19. The method of any one of claims 13 to 18, wherein the population of cells comprises one or more of endothelial cells, monocytes, or fibroblasts.

20. A method of downregulating an innate immune response in a subject comprising administering an effective amount of an antagonist of the interaction between DAPK and stimulator of interferon genes protein (STING) pathway.

21. The method of claim 20, wherein the antagonist is a protein, peptide, small molecule, antibody, bispecific antibody, antibody derivative, ligand mimetic, nucleic acid, or pharmaceutical composition.

22. The method of claim 20, wherein the antagonist is an anti-DAPK1 antibody.

23. The method of claim 20, wherein the antagonist is:

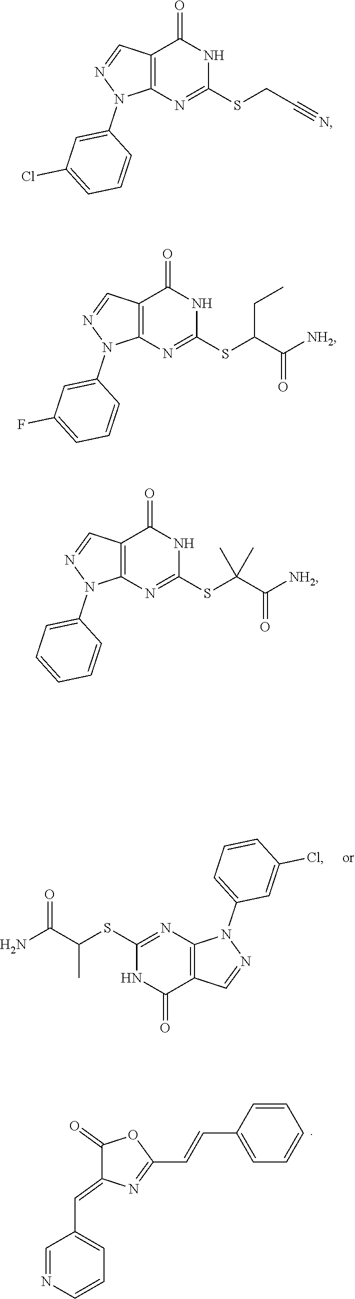 [SMILES: N#CCSc1nc2c(cnn2-c2cccc(Cl)c2)c(=O)[nH]1 ; CCC(Sc1nc2c(cnn2-c2cccc(F)c2)c(=O)[nH]1)C(N)=O ; CC(C)(Sc1nc2c(cnn2-c2ccccc2)c(=O)[nH]1)C(N)=O ; CC(Sc1nc2c(cnn2-c2cccc(Cl)c2)c(=O)[nH]1)C(N)=O ; O=C1OC(/C=C/c2ccccc2)=NC/1=C\c1cccnc1]

24. The method of claim 20, wherein the antagonist downregulates, inhibits, or knocks out DAPK expression.

25. The method of claim 24, wherein the antagonist is an inhibitory nucleic acid, optionally an siRNA or shRNA.

26. The method of any one of claims 20 to 25, wherein the antagonist is an antagonist of the interaction between DAPK and TBK1 and/or STING.

27. The method of any one of claims 20 to 26, wherein the subject is a mammal.

28. The method of claim 27, wherein the mammal is a human.

29. The method of any one of claims 20 to 28, wherein the DAPK is DAPK1, DAPK2, or DAPK3.

30. The method of any one of claims 20 to 29, wherein the subject has an inflammatory or autoimmune disease.

31. The method of claim 30, wherein the inflammatory or autoimmune disease is polymyositis, vasculitis syndrome, giant cell arteritis, Takayasu arteritis, relapsing, polychondritis, acquired hemophilia A, Still's disease, adult-onset Still's disease, amyloid A amyloidosis, polymyalgia rheumatic, Spondyloarthritides, Pulmonary arterial hypertension, graft-versus-host disease, autoimmune myocarditis, contact hypersensitivity (contact dermatitis), gastro-esophageal reflux disease, erythroderma, Behcet's disease, amyotrophic lateral sclerosis, transplantation, rheumatoid arthritis, juvenile rheumatoid arthritis, malignant rheumatoid arthritis, Drug-Resistant Rheumatoid Arthritis, Neuromyelitis optica, Kawasaki disease, polyarticular or systemic juvenile idiopathic arthritis, psoriasis, chronic obstructive pulmonary disease (COPD), Castleman's disease, asthma, allergic asthma, allergic encephalomyelitis, arthritis, arthritis chronica progrediente, reactive arthritis, psoriatic arthritis, enterophathic arthritis, arthritis deformans, rheumatic diseases, spondyloarthropathies, ankylosing spondylitis, Reiter syndrome, hypersensitivity (including both airway hypersensitivity and dermal hypersensitivity), allergies, systemic lupus erythematosus (SLE), cutaneous lupus erythematosus, erythema nodosum leprosum, Sjögren's Syndrome, inflammatory muscle disorders, polychondritis, Wegener's granulomatosis, dermatomyositis, Steven-Johnson syndrome, chronic active hepatitis, myasthenia gravis, idiopathic sprue, autoimmune inflammatory bowel disease, ulcerative colitis, Crohn's disease, Irritable Bowel Syndrome, endocrine ophthalmopathy, scleroderma, Grave's disease, sarcoidosis, multiple sclerosis, primary biliary cirrhosis, vaginitis, proctitis, insulin-dependent diabetes mellitus, insulin-resistant diabetes mellitus, juvenile diabetes (diabetes mellitus type I), autoimmune haematological disorders, hemolytic anemia, aplastic anemia, pure red cell anemia, idiopathic thrombocytopenia (ITP), autoimmune uveitis, uveitis (anterior and posterior), keratoconjunctivitis sicca, vernal keratoconjunctivitis, interstitial lung fibrosis, glomerulonephritis (with and without nephrotic syndrome), idiopathic nephrotic syndrome or minimal change nephropathy, inflammatory disease of skin, cornea inflammation, myositis, loosening of bone implants, metabolic disorder, atherosclerosis, dislipidemia, bone loss, osteoarthritis, osteoporosis, periodontal disease of obstructive or inflammatory airways diseases, bronchitis, pneumoconiosis, pulmonary emphysema, acute and hyperacute inflammatory reactions, acute infections, septic shock, endotoxic shock, adult respiratory distress syndrome, meningitis, pneumonia, cachexia wasting syndrome, stroke, herpetic stromal keratitis, dry eye disease, iritis, conjunctivitis, keratoconjunctivitis, Guillain-Barre syndrome, Stiff-man syndrome, Hashimoto's thyroiditis, autoimmune thyroiditis, encephalomyelitis, acute rheumatic fever, sympathetic ophthalmia, Goodpasture's syndrome, systemic necrotizing vasculitis, antiphospholipid syndrome, Addison's disease, pemphigus vulgaris, pemphigus foliaceus, dermatitis herpetiformis, atopic dermatitis, eczematous dermatitis, aphthous ulcer, lichen planus, autoimmune alopecia, Vitiligo, autoimmune hemolytic anemia, autoimmune thrombocytopenic purpura, pernicious anemia, sensorineural hearing loss, idiopathic bilateral progressive sensorineural hearing loss, autoimmune polyglandular syndrome type I or type II, immune infertility and immune-mediated infertility.

32. The method of any one of claims 20 to 31, wherein the antagonist is specifically administered to one or more of endothelial cells, monocytes, or fibroblasts.

33. A method of decreasing type I interferon (IFN-I) expression in a population of cells comprising administering an effective amount of an antagonist of the interaction between DAPK and stimulator of interferon genes protein (STING) pathway to a population of cells.

34. The method of claim 33, wherein the antagonist is a protein, peptide, small molecule, antibody, bispecific antibody, antibody derivative, ligand mimetic, nucleic acid, or pharmaceutical composition.

35. The method of claim 33, wherein the antagonist is an anti-DAPK1 antibody.

36. The method of claim 33, wherein the antagonist is:

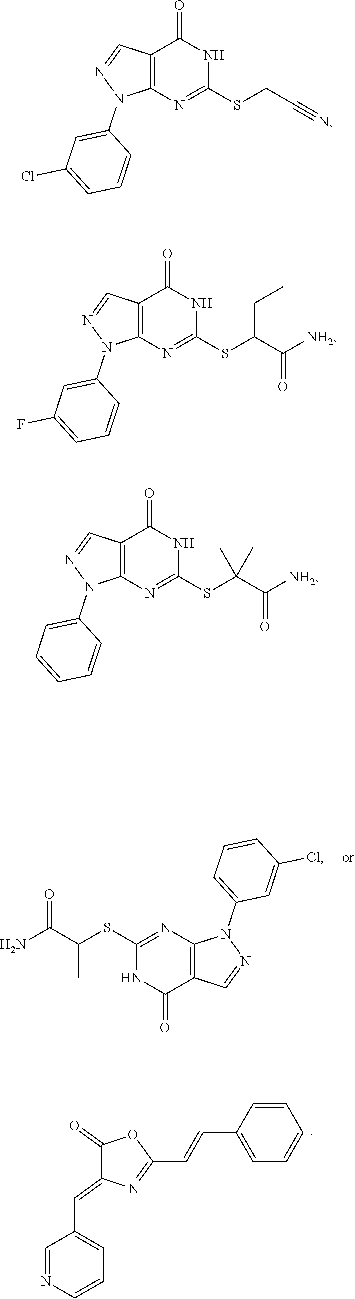 [SMILES: N#CCSc1nc2c(cnn2-c2cccc(Cl)c2)c(=O)[nH]1 ; CCC(Sc1nc2c(cnn2-c2cccc(F)c2)c(=O)[nH]1)C(N)=O ; CC(C)(Sc1nc2c(cnn2-c2ccccc2)c(=O)[nH]1)C(N)=O ; CC(Sc1nc2c(cnn2-c2cccc(Cl)c2)c(=O)[nH]1)C(N)=O ; O=C1OC(/C=C/c2ccccc2)=NC/1=C\c1cccnc1]

37. The method of claim 33, wherein the antagonist downregulates, inhibits, or knocks out DAPK expression.

38. The method of claim 37, wherein the antagonist is an inhibitory nucleic acid, optionally an siRNA or shRNA.

39. The method of any one of claims 33 to 38, wherein the antagonist is an antagonist of the interaction between DAPK and TBK1 and/or STING.

40. The method of any one of claims 33 to 39, wherein the DAPK is DAPK1, DAPK2, or DAPK3.

41. The method of any one of claims 33 to 40, wherein the population of cells comprises one or more of endothelial cells, monocytes, or fibroblasts.
